SEQUENCE LISTING (2 sequences)

SEQ ID NO: 1 - RNA, 58 nt, Artificial Sequence
  chemically synthesized
ccgggcuaagucuuugcacaguaaacucgaguuuacugugcaaagacuuagcuuuuug

SEQ ID NO: 2 - RNA, 58 nt, Artificial Sequence
  chemically synthesized
ccggccacaugguuaauaagagaaucucgagauucucuuauuaaccaugugguuuuug

(19) United States
(12) Patent Application Publication
(10) Pub. No.: US 2010/0291188 A1
(43) Pub. Date: Nov. 18, 2010

(54) Periostin Inhibitory Compositions for Myocardial Regeneration, Methods of Delivery, and Methods of Using Same

(75) Inventors: RUSSELL A. NORRIS, SUMMERVILLE, SC (US); ROBERT G. GOURDIE, CHARLESTON, SC (US); MICHAEL P. O'QUINN, CHARLESTON, SC (US); ROGER R. MARKWALD, MT. PLEASANT, SC (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: MUSC FOUNDATION FOR RESEARCH DEVELOPMENT, CHARLESTON, SC (US)

(21) Appl. No.: 12/631,655

(22) Filed: Dec. 4, 2009

Related U.S. Application Data
(60) Provisional application No. 61/120,053, filed on Dec. 4, 2008.

(51) Int. Cl. A61K 9/127; A61K 35/76; C12Q 1/18; C12Q 1/70; G01N 33/00; A61K 31/7088; A61K 38/00

(52) U.S. Cl. 424/450; 424/93.6; 435/32; 435/5; 800/3; 514/44 R; 514/44 A; 514/1.1

(57) ABSTRACT

We claim the compositions of matter and methods described herein.

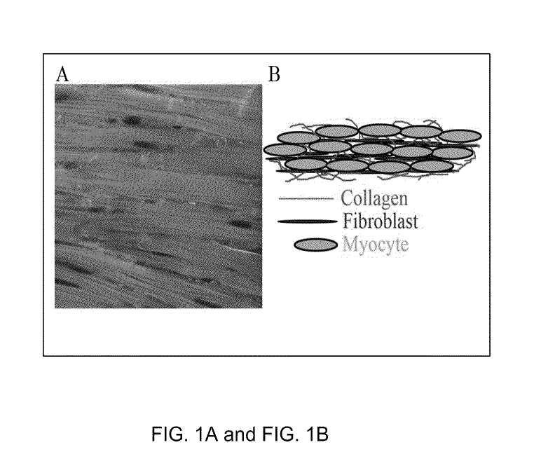

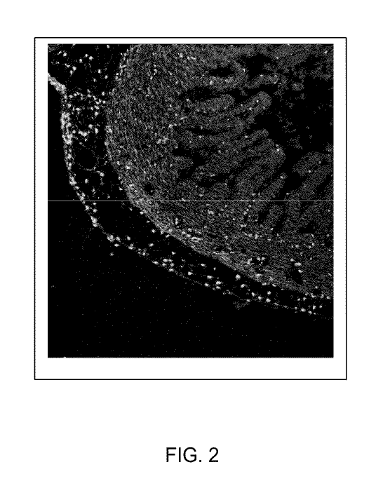

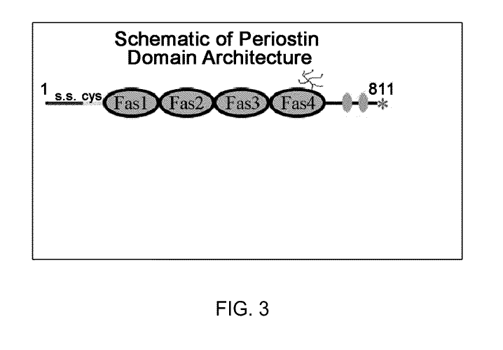

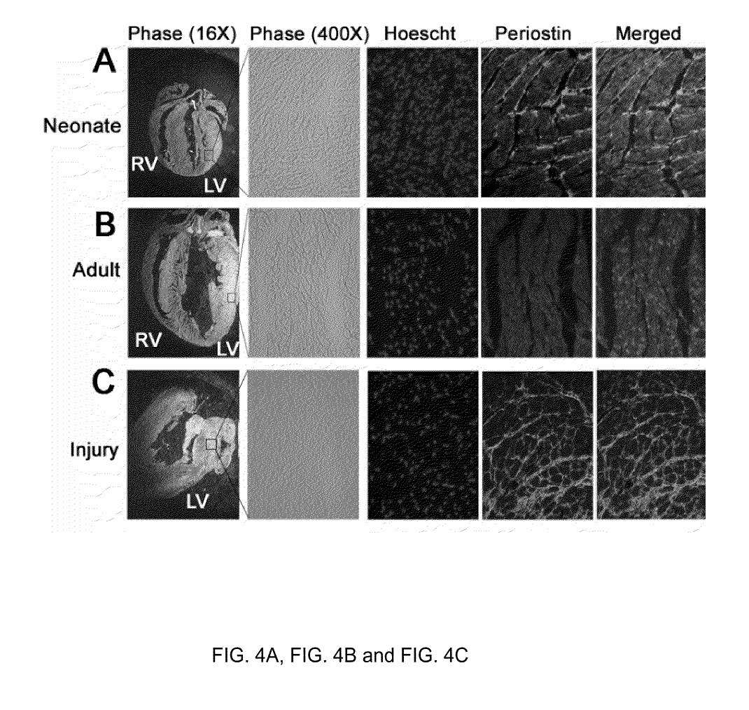

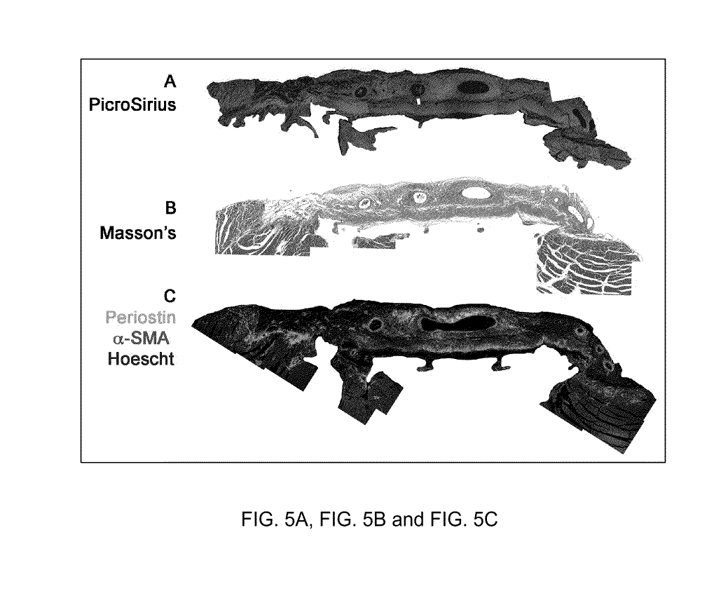

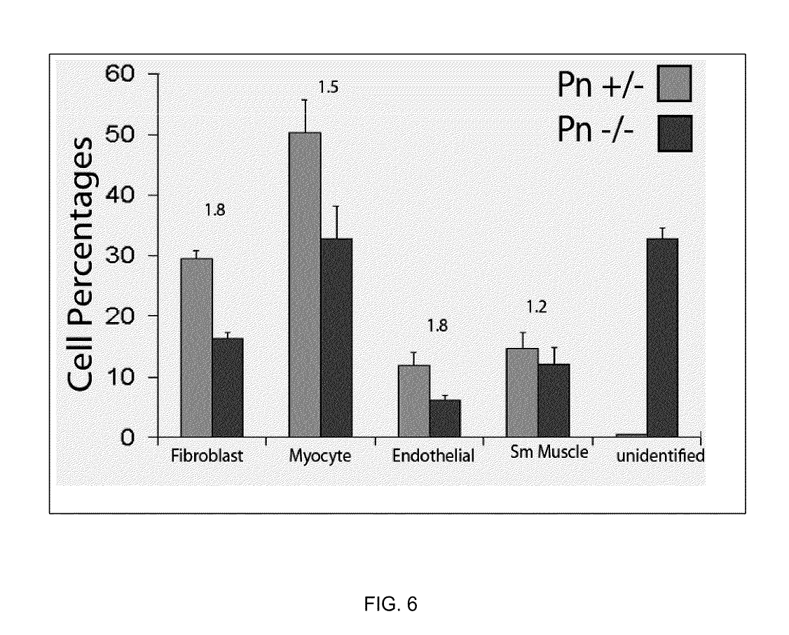

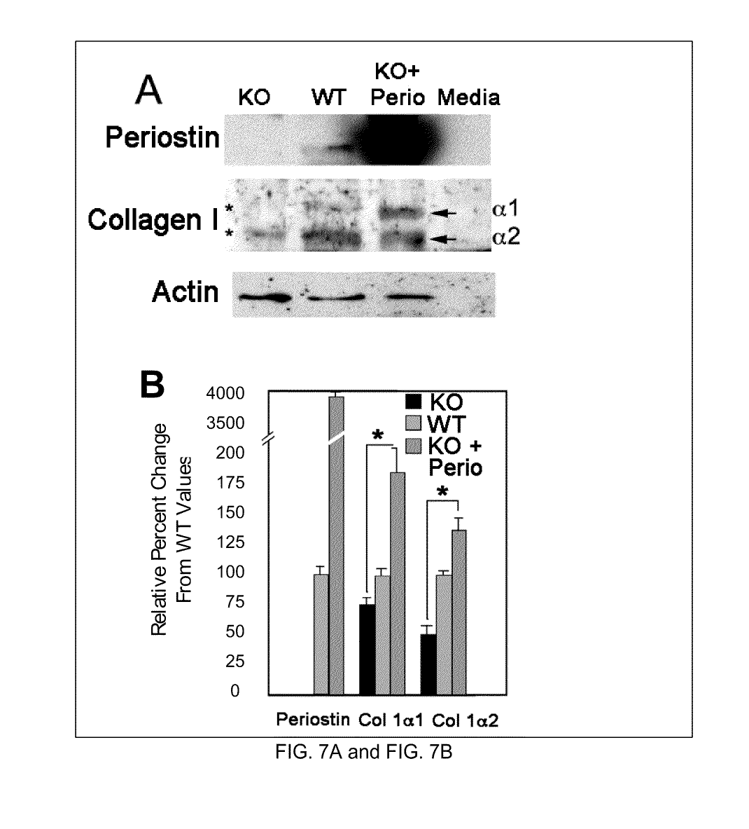

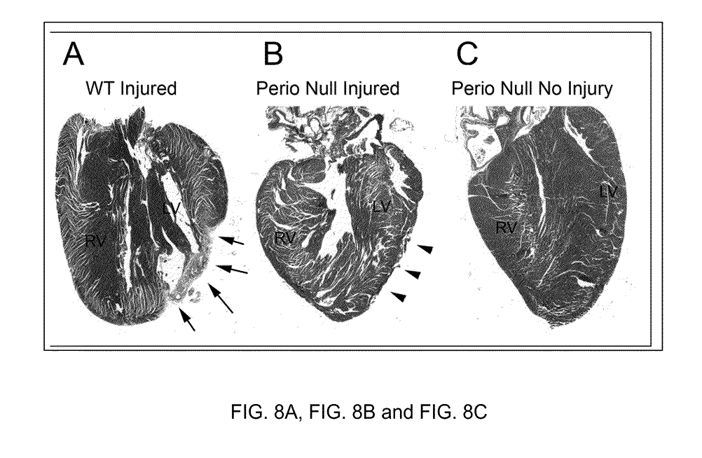

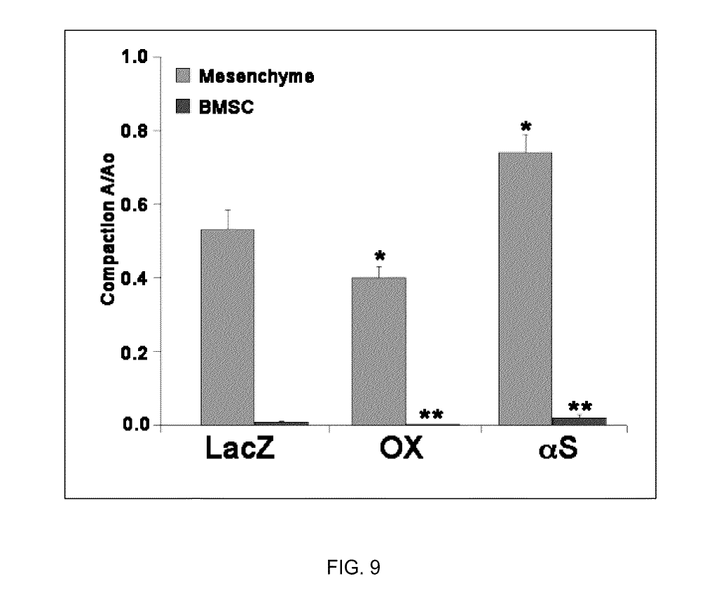

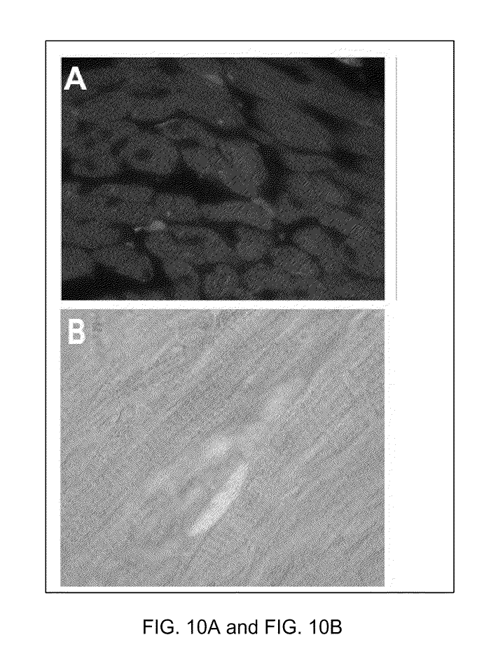

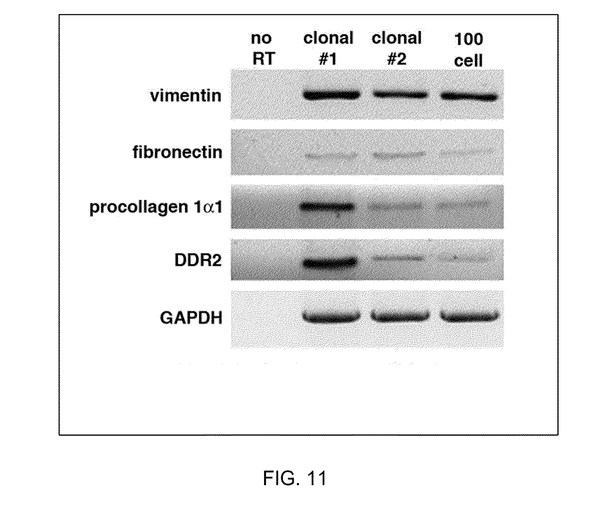

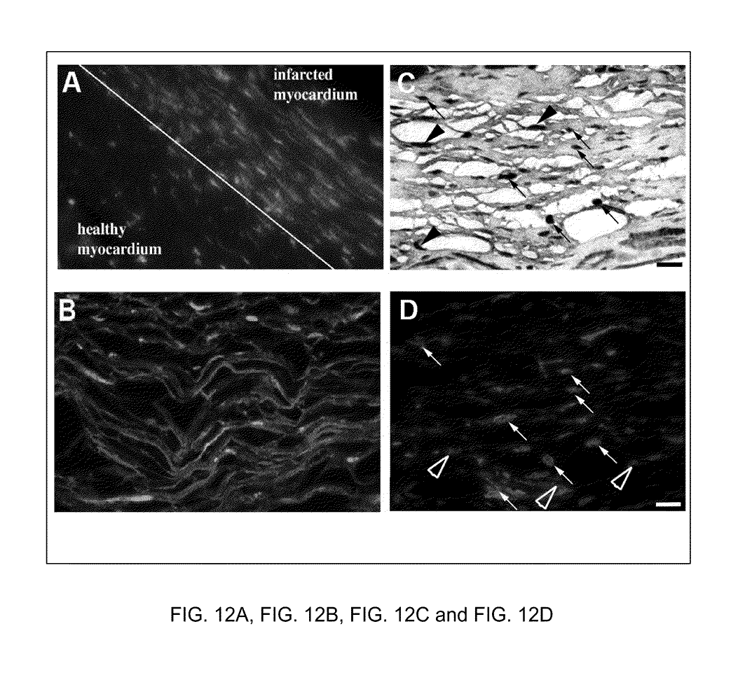

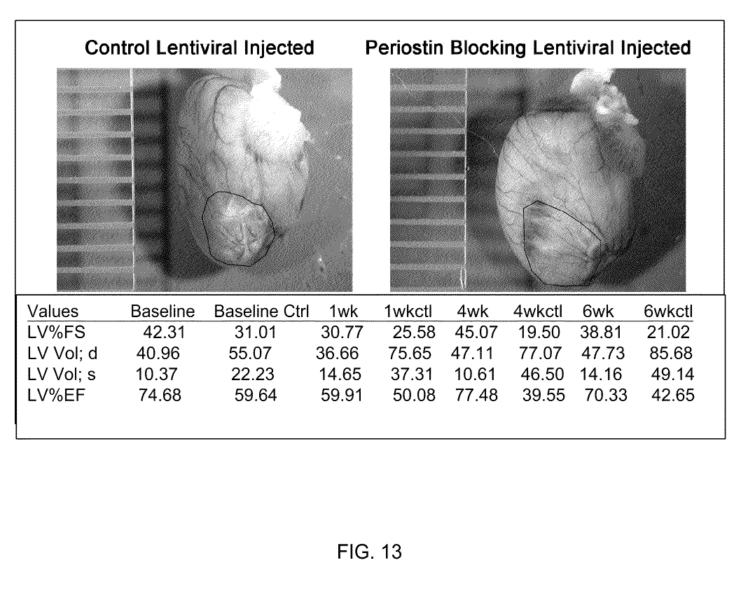

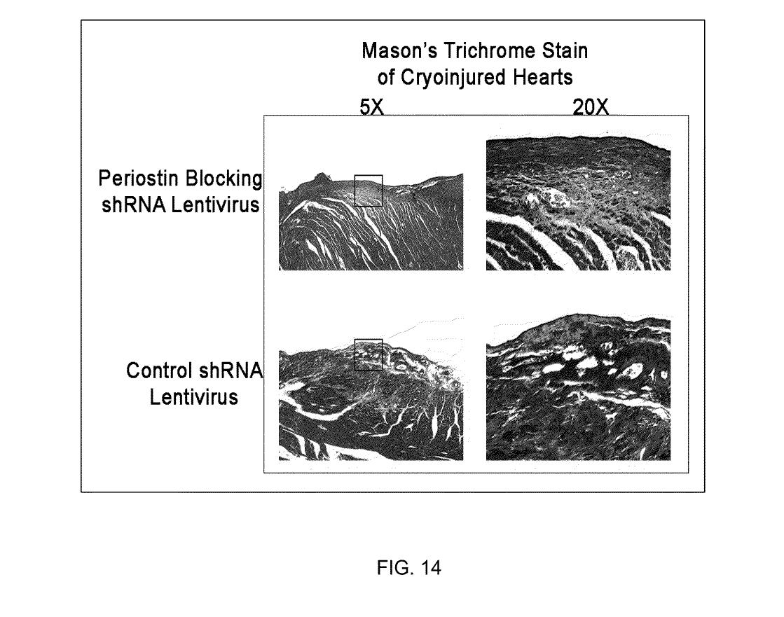

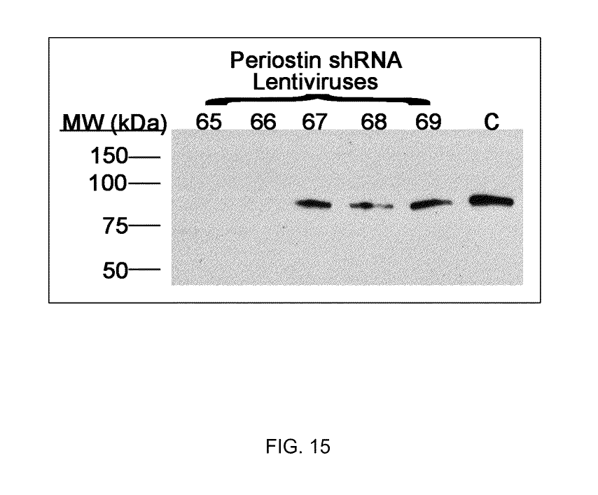

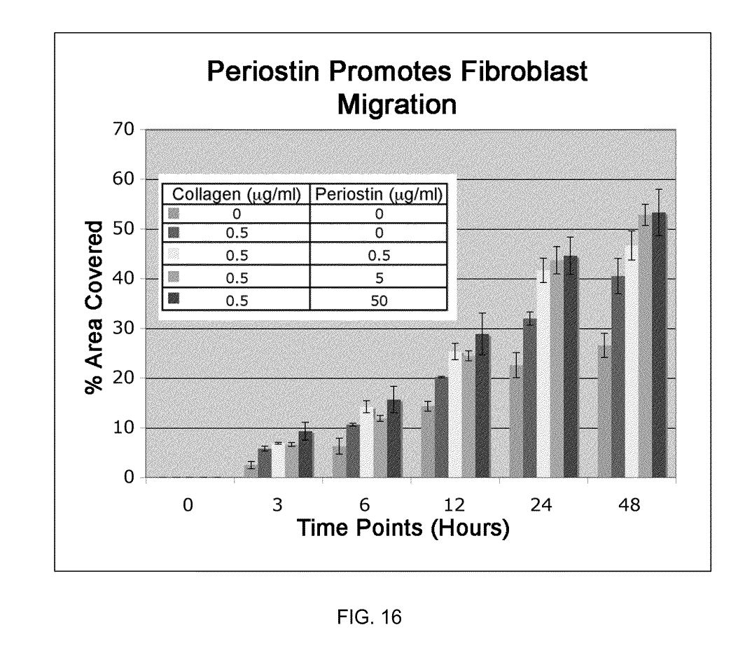

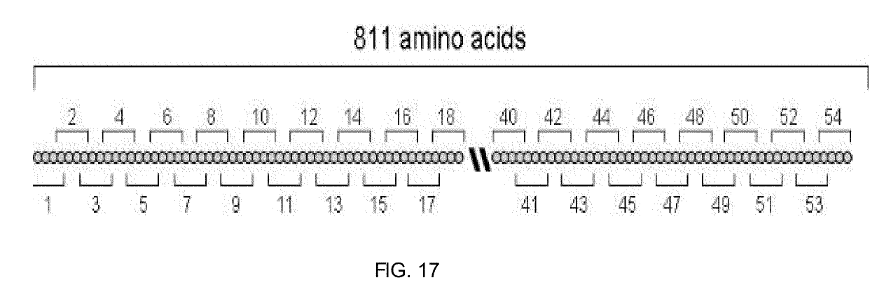

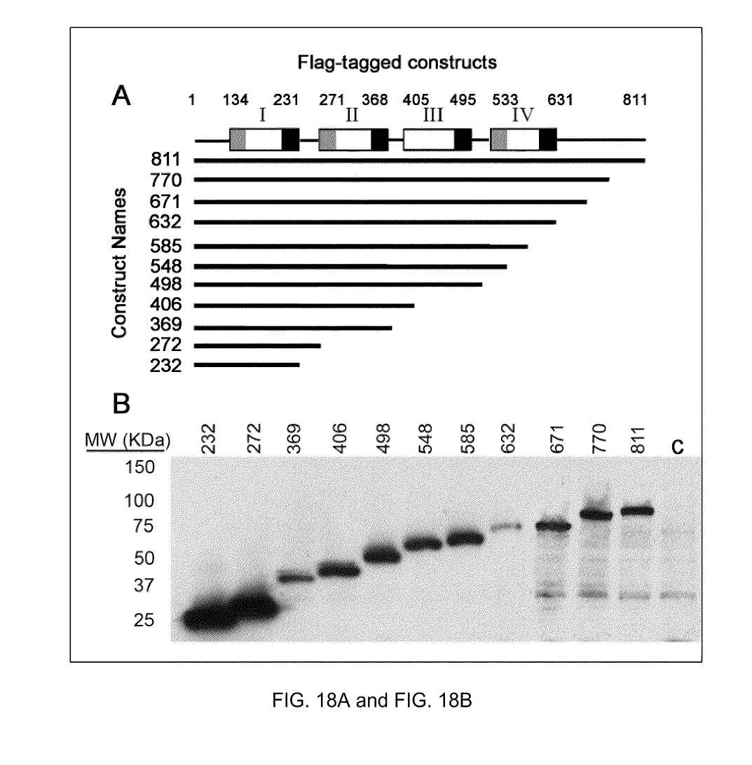

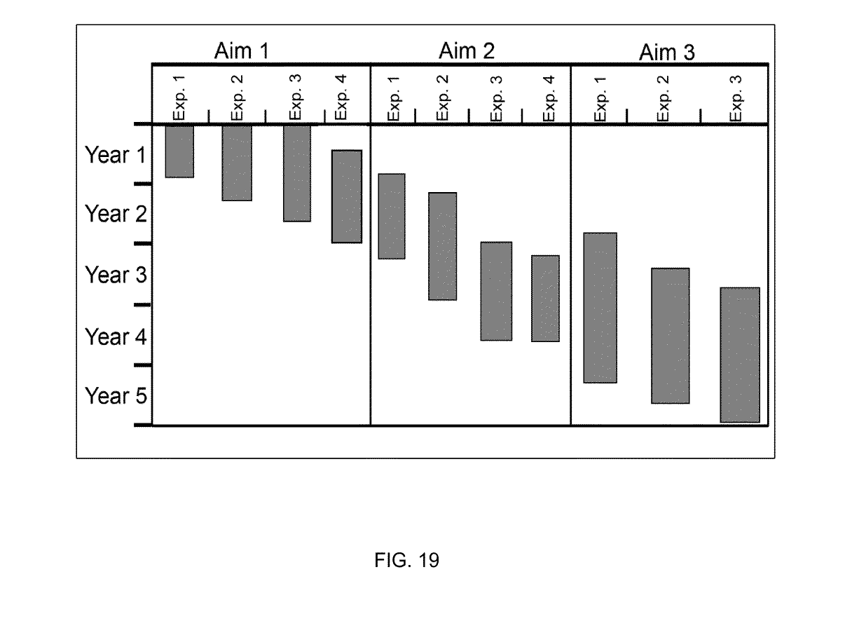

### CROSS-REFERENCE TO RELATED APPLICATIONS

This application claims benefit of U.S. Provisional Application No. 61/120,053, filed Dec. 4, 2008. Application No. 61/120,053, filed Dec. 4, 2008, is hereby incorporated herein by reference in its entirety.

### BRIEF DESCRIPTION OF THE INVENTION

Coronary heart disease is the leading cause of death in the America. While one American death in every five annually is caused by coronary heart disease, treatment options remain limited. Once injury to the heart occurs, the cardiac healing process is dominated by scar tissue formation. This leads to a diminished function of the damaged heart as a blood pump and increased risk of cardiac arrhythmia and sudden death. Embodiments of the invention comprise compositions of matter and a method of using same that encourages the regeneration of heart muscle rather than scar tissue following a heart attack.

Embodiments of the invention comprise a method for modifying the behavior of stem cells in vivo to promote re-growth and/or repopulation of myocytes following heart attack. By blocking a protein, scars that normally form in place of damaged myocardial tissue are reduced and, in turn, there may be: (i) infiltration of new myocytes, (ii) proliferation of resident myocytes, (iii) differentiation of stem cells into new myocytes. Embodiments of the invention comprise compositions of matter and the delivery thereof. Embodiments of the invention comprise a novel lentivirus which blocks the expression of a gene called periostin.

Additional embodiments of the invention are a delivery system comprising performing a bone marrow injection of the therapeutic (either prior to, during, or shortly after a heart attack occurs) directly into the medullary canal of both femurs for the purpose of infecting as many bone marrow stem cells as possible. We have demonstrated that bone marrow derived stem cells are activated and mobilized following injury and are subsequently carried, through systemic circulation, to these injury sites. Once there, they differentiate into fibroblasts which are foundational for scar progression. However, by blocking periostin with composition of matter embodiments of the invention, the stem cells are unable to properly and/or completely differentiate into fibroblasts thereby resulting in an abrogation of scar formation and repopulation of the wounded area with functional myocardial tissue.

### BRIEF DESCRIPTION OF DRAWINGS

FIG. B-1

FIG. B-1: Myocardial Organization. (A) Confocal image of fibroblasts stained with DDR2 (blue) lying within the collagen network (red) between layers of myocytes. (B) Diagramatic representation of (A).

FIG. B-2

FIG. B-2: Contribution of EPDCs to the ventricular myocardium. Sagittal section of E16.5 WT1-EGFP transgenic mouse heart showing EPDC cells invading the ventricular wall.

FIG. B-3

FIG. B-3: Schematic of Periostin Protein. Fasciclin domains (Fas1-4), signal sequence (S.S.), cysteine rich domain (Cys), heparin binding domain (green ovals), putative glycosylation site (branch structure), and stop codon (red asterisk) are depicted.

FIG. C-1

FIG. C-1: Periostin expression is re-activated during myocardial injury in the adult heart. (A)

Staining for periostin in a 2 week old neonate showing robust expression in the left ventricular myocardium. (B) Periostin expression has greatly diminished in the adult being weekly expressed by cardiac fibroblasts. (C) Following cryoinjury, periostin expression is reactivated in the border zone. All images were taken at the same settings.

Hoescht-nuclear stain

FIG. C-2

FIG. C-2: Periostin expression after myocardial injury correlates with collagen deposition. (A) Picrosirius staining showing prevalence of bright orange staining signifying presence of thick collagen fibrils. (B) Masson's staining showing extensive collagen staining (blue) in the scar. (C) IHC for Periostin and □-SMA showing significant overlap between periostin and collagen deposition. Sections are sister slides through the scar. Hosecht-nuclear stain.

FIG. C-3

FIG. C-3: Cellular differentiation defects in the periostin null mice. Week 12 hearts were prepared for FACS sorting using antibody markers for fibroblasts (DDR2), smooth muscle (α-SMA), myocytes (α-MHC), and endothelial cells (PECAM/CD31). In the periostin−/− mice a large percentage of unidentifiable cells exist. Numbers represent fold changes.

FIG. C-4

FIG. C-4: Periostin regulates collagen I synthesis. Mesenchyme from periostin null mice were placed in hanging drop cultures and incubated with either purified periostin protein (10 □g/ml) or PBS. Mesenchyme from wild-type mice were used as control tissues. After 7 days, tissues were analyzed for periostin and collagen expression by Western analysis. (A) Periostin null mesenchyme exhibit low levels of collagen I□1 and I□2. Addition of periostin to the culture medium induces expression of collagen I□1 and I□2. Actin is used for protein normalization (B) Graphical representation of Western analyses presented in panel A obtained from densitometric analyses using NIH image software. Values obtained were compared against wild-type values (baseline) and represented as relative percent change. * denotes p<0.05

FIG. C-5

FIG. C-5: Periostin null mice exhibit reduced scar formation following cardiac injury. Cryoablation injuries were given to age-matched wild-type and periostin null mice and followed for 8 weeks. After 8 weeks, hearts were extracted, fixed, sectioned, and stained for Masson's trichrome. (A) Wild-type mice exhibited pronounced scar formation (arrows) whereas the same injury in the periostin null mice (B) resulted in a significantly reduced scar (arrow-heads) (C) Control, non-injured heart.

FIG. C-6

FIG. C-6: Periostin promotes collagen compaction. Cushion mesenchyme and adult bone marrow stem cells (BMSC) were tested for their ability to compact collagen gels. Cells were infected with either a control (LacZ), periostin over-expressing (OX) or periostin blocking (□S) adenovirus. Cells were allowed to freely compact collagen gels for 7 days after which final area measurements were calculated. By expressing more periostin, both cell types exhibited an increase in compaction whereas blocking periostin reduces this compaction. * and ** denotes p<0.05 for mesenchyme and BMSCs, respectively.

FIG. C-7

FIG. C-7. HSC-derived cells are detected in recipient mouse myocardium. (A) EGFP+ interstitial fibroblasts (green) are detected intercalated between the myocytes in the ventricular myocardium of mice transplanted with EGFP+ bone marrow HSCs. (B) Higher magnification of A.

FIG. C-8

FIG. C-8: Bone marrow-derived fibroblasts express fibroblast markers in vitro. RT-PCR analysis of EGFP⁺ BM cells in vitro. Two separate clones (C1, C2) express fibroblast specific transcripts.

FIG. C-9

FIG. C-9: EGFP/Periostin/pro-collagen I expression in adult cardiac tissues. (A) EGFP engraftment after infarct; increased presence of EGFP+ HSC derived cells (green) in the scar vs. border zones. (B) HSC-derived (EGFP⁺) cells (green) are surrounded by periostin (red) in the infarcted myocardium. (C) Pro-collagen I□I RNA expression in the infarct region. (D). IHC for EGFP in the adjacent 3 □m section shows a subpopulation of EGFP⁺ cells expressing pro-collagen I□I mRNA. Arrows indicate HSC-derived fibroblasts (EGFP+/col I□I+). Solid arrowheads indicate non-HSC fibroblasts (EGFP−/col I□I+), hollow arrowheads indicate HSC-derived non-myofibro-blastic cells (EGFP+/col I□I−). Mag.=400×

FIG. C-10 FIG. C-10: Bone marrow injection of Periostin inhibitory shRNA lentiviruses reduces scar formation in vivo. shRNA lentiviruses were directly injected into the femur medullary canals of wild-type mice followed by cryoinjury of the hearts. Experimental animals (using periostin blocking lentiviruses) exhibit a significant reduction in scar size and enhanced cardiac function (compared to control hearts) as determined by gross whole mount analyses and echocardiography four-six weeks post-injury, respectively. Lines depict initial cryoinjury location

FIG. C-11

FIG. C-11: Bone marrow injection of Periostin inhibitory shRNA lentiviruses reduces scar formation in vivo and potentiates myocardial repopulation. Masson's trichrome stain of infected hearts depicted in FIG. C-10 showing a smaller scar with significant myocardial repopulation in the periostin blocked hearts suggesting myocardial regeneration. Boxed regions in the 5× panel are depicted in the 40× panel. Blue-collagen, Red-cardiac muscle

FIG. D-1

FIG. D-1: Novel shRNA Lentiviruses block endogenous periostin expression. Western analysis for periostin expression of shRNA lentiviral infected adult cardiac fibroblasts. Numbers 65 and 66 completely blocked periostin translation. Equal amounts of media fraction were tested. C-control non-infected media.

FIG. D-2

FIG. D-2: Varying amounts of periostin and collagen coated dishes were assayed for their ability to promote wild-type fibroblast migration. Data demonstrate that there is a titrating affect of periostin in combination with collagen to stimulate migration of these cells.

FIG. D-3

FIG. D-3: Schematic of synthesized periostin peptides. Green balls represent 5 amino acids along the periostin protein with brackets and numbers corresponding to each of the peptides.

FIG. D-4

FIG. D-4: Periostin carboxyl truncation mutants. (A) Schematic of periostin and design of truncation mutants. Each truncation mutant is generated with a FLAG epitope tag at the C-terminus. Fasciclin domains are indicated by Roman numerals. Within each fasciclin domain are two regions exhibiting high homology between periostin and other family members: grey boxes-YH domains, black boxes-H2 domains. Amino acids positions are depicted at the top. (B) Western analysis of each of the 10 truncation mutants and full length (811). C-empty vector transfected control

FIG. D-5

FIG. D-5: Time-line for completion of the project. Each aim is divided into the main experiments proposed. Gray bars represent the anticipated start time and completion of each experiment. Total completion of this project is anticipated during the 5^{th} year.

### BACKGROUND OF THE INVENTION

It has become increasingly clear that cardiac development is not complete during intrauterine life. Rapid enlargement and adaptive (or physiological) “remodeling” in humans occurs during the first three weeks of the postnatal period to bridge embryonic/fetal development with the fully defined/mature mammalian heart. This postnatal response is necessary to comply with the increased systolic pressures of the growing neonate. The forced adaptation of the myocardial wall is accomplished largely in part through the increase in the cardiac fibroblast population and their localized secretion of critical stabilizing matrix proteins such as collagen I. Collectively, this remodeling event results in an increase in ventricular wall thickness and stiffness (i.e. tensile strength). In the mouse, the adaptive response peaks during the first two weeks post-natally as the mature cardiac phenotype is fully established by 30 days.

However, cardiac remodeling can be rekindled during adult life in response to changes in the environment (e.g. pressure overload or ischemic injury). Unlike neonatal remodeling, adult remodeling can progressively evolve into a pathological response resulting in heart failure and death. Although nearly 1 in 5 adults will develop congestive heart failure, relatively little is known regarding the role of the cardiac fibroblast in the progression of this disease. Current “standard of care” treatment focus on improving the symptoms and preventing the progression of heart disease. Reversible causes of the heart failure also need to be addressed: (e.g., ingestion, anemia, hypertension). Treatments include lifestyle and pharmacological modalities.

There is a significant evidence—practice gap in the treatment of CHF; particularly the underuse of and aldosterone antagonists which have been shown to provide mortality benefit. Treatment of CHF aims to relieve symptoms, to maintain a state (normal fluid level in the circulatory system), and to improve by delaying progression of heart failure and reducing cardiovascular risk. Some drugs which increase heart function, such as the positive inotrope, lead to increased mortality, and are contraindicated.^{i ii}

Embodiments of the invention regenerate new myocytes in vivo following a heart attack, and as such, enhance and maintain cardiac integrity, function, and viability following cardiac injury.

Neonatal cardiac remodeling can be rekindled during adult life in response to changes in the environment (e.g. pressure overload or ischemic injury). Unlike neonatal remodeling, adult remodeling progressively evolves into a pathological response resulting in heart failure and death. Although nearly 1 in 5 adults will develop congestive heart failure, relatively little is known regarding the role of the cardiac fibroblast in the progression of this disease. We have shown that periostin, a fibroblast specific gene, is a key regulator of fibroblast function during normal (neonatal) and pathological (adult injury) myocardial remodeling.

B.1 Heart Development Extends Beyond Intrauterine Life.

The development and maturation of the mammalian heart is not complete at birth. Within the first three weeks after birth there is an increase in systolic force (pressure overload) and end-diastolic wall stress (volume overload) that stimulates an overall tissue remodeling¹⁻⁴. These biomechanical stimuli result in cardiomyocyte hypertrophy (in place of proliferation) and nonmyocyte hyperplasia. It is both the progressive hypertophy of myocytes and hyperplasia of cardiac fibroblasts that result in a marked increase in ventricular wall size and strength. During this neonatal period, cardiac fibroblasts secrete and organize a collagenous network that envelops myocytes as an endomyseal-like, collagenous network^{5,6} (FIG. B-1). This organization provides for (i) “contact-signaling” between myocytes and fibroblasts (heterotypic interactions), (ii) fibroblasts themselves (homotypic interactions), or (iii) cell-ECM contacts⁷⁻¹⁰. Each of these three forms of “contact-signaling” are essential for preparing the postnatal myocardial wall to structurally adapt and remodel in response to increases in blood pressure after birth. Surprisingly, while there is considerable literature on the structure and function of neonatal cardiac myocytes, much less is known regarding the role(s) of fibroblasts during postnatal development. However, we propose that the cardiac fibroblast and its secreted matrix is of hallmark importance for promoting myocardial remodeling and maintaining the structural integrity of the heart during the cardiac cycle after birth. Of significance, a more thorough understanding and appreciation for cardiac fibroblast function during neonatal development is essential since it is these developmental programs that are reawakened during adult cardiac pathologies such as myocardial infarction and cardiomyopathies.

B.2 The Origin(s) of Cardiac Fibroblasts

One reason why the role of fibroblasts in normal or later pathological remodeling of the heart is not well understood is that their developmental origin(s) is not clear. Fibroblasts in the heart are thought to arise from various sources during development. In the embryonic heart, fibroblasts arise from the differentiation of cells derived from the proepicardial organ in addition to the differentiation of endocardial cushion mesenchymal cells. In the post-natal heart, bone marrow stem cells appear to give rise to circulating progenitor cells (hematopoeitc stem cells) which differentiate into cardiac fibroblasts.

The Proepicardial Organ: The PEO is a sac-like vascular structure that is derived from the coelomic mesothelium located at the venous inlet (sinuatrial) pole of the heart^{11,12}. The PEO gives origin to cells that migrate as an epithelium over the surface of the entire heart to form the embryonic epicardium. The epicardium, in turn, undergoes a cell autonomous epithelial to mesenchymal transformation (EMT) to form epicardial derived cells or EPDCs that accumulate as mesenchyme between the epicardium and myocardium. During the late embryonic and fetal period, the EPDCs invade the atrial and ventricular walls and functionally interact with cardiomyocytes to establish a compact myocardium¹¹⁻¹⁶. Failure of EPDCs to invade the myocardium and interact with developing cardiomyocytes results in lethality due to the failure to form a compact myocardium. FIG. B-2 was prepared in collaboration with Dr. John Burch (Fox Chase Cancer Center) to illustrate the invasion of EPDCs into the ventricular myocardium as revealed by their expression of EGFP driven by the Wilms Tumor promoter. Thus, from the beginning of cardiac embryogenesis, there is an interaction between the fibroblasts (or their progenitors) and myocardial cells that is necessary for myocardial growth, survival and function.

While EPDCs are an immediate progenitor of cardiac fibroblasts, it is important to recognize that they are also multipotential cells. They have potential to differentiate in vitro into endothelial cells, smooth muscle and cardiomyocytes^{11, 14-16}. However, in vivo, during normal embryonic life, most EPDCs within the wall of the heart progressively differentiate into fibroblasts which oscillate or transdifferentiate between two phenotypes: one is spindle-shaped; the other is more rounded and sometimes called a “myofibroblast” because it expresses □-smooth muscle actin. Over time, expression of smooth muscle □-actin is suppressed and most EPDC derivatives assume a more spindle-shaped phenotype.

Recruited fibrogenic progenitor cells: As indicated above, we have found that other mesenchymal-like cells are also added to the postnatal heart^{17, 18}. As described in Section C, the progeny of a single (EGFP+) donor hematopoietic stem cell (HSC) injected to repopulate the bone marrow of a lethally irradiated adult mouse host will also give rise to EGFP+ cells in the adult heart. Preliminary findings in mice indicate that EGFP+ cells differentiate into cardiac fibroblasts¹⁷⁻¹⁹. Current controversy regarding the lineage potential of any given “stem cell” may be due to the fact that most studies evaluating stem cell potential invariably use mixed cell populations²⁰⁻²³. Thus, we believe that the definitive assignment of fate (or lineage) must be based on the analysis of a single donor cell's ability to achieve long-term organ engraftment and multi-lineage hematopoietic reconstitution of bone marrow when injected into the tail vein of an irradiated mouse host. Bone marrow reconstitution of lethally irradiated mice with a single HSC is no small feat; thus, very few laboratories have successfully performed the procedure. Dr. Visconti, a co-investigator on this application was trained in the laboratory of Dr. Makio Ogawa (MUSC) who pioneered single cell transplantation strategies utilizing hematopoietic stem cells derived from mice that express EGFP on a universal promoter²⁴. This permits the full potential of a single HSC to be evaluated in vivo (see Section C). Using this system, Dr. Visconti has published five studies describing the potential of HSCs to give rise to cell types not traditionally attributed to hematopoietic stem cells, e.g. fibroblasts or myofibroblasts.

Whatever their origin (EPDCs, HSCs, or endocardial cushion mesenchyme), it is important to recognize that undifferentiated fibroblastic progenitor cells are present in normal newborn and adult hearts (see Section C). In the periostin null animals, over one-third of the total cell population isolated from adult hearts remain mesenchymal-like and do not express any differentiation markers. In part, for this reason, we focus on periostin as a candidate regulatory gene that directs differentiation of cardiac derived, mesenchymal progenitor cells into a fibroblastic lineage.

B3. Periostin is a Fasciclin Related Gene.

The periostin gene was initially cloned from a mouse calvarial cell line (MC3T3-L1) and shown to promote adhesion and migration of these cells in culture²⁵ . The encoded protein has a molecular weight of 90 kDa and, based on amino acid sequence similarities, is most highly related to the ancestral fasciclin gene in Drosophila^{26, 27}. The protein has a signal sequence (targeting it for secretion), four coiled fasciclin like repeats, an amino terminal cysteine rich region and putative heparin binding domains present in the carboxyl tail (FIG. B-3). RT-PCR, Western analysis, and genomic sequencing have revealed that at least 6 carboxyl splice variants may be produced from the periostin locus²⁸⁻³¹. It is an evolutionarily conserved protein with chick and zebrafish periostin being 65% homologous to mouse and 70% to human (73% to rat) at the amino acid level^{29, 32}. Periostin is one of four known mammalian genes that encode fasiclin domains. The other fasciclin genes are: TGFβ-Induced Gene-Human clone 3 (a.k.a βigH3), as well as stabilin 1 and 2. βIG-H3 shares 49% overall amino acid homology (70% homology in the fasiclin domain) with periostin whereas the stabilin proteins are significantly more divergent.

Periostin expression during development: Periostin expression has been extensively examined during embryonic and fetal development^{28, 33-37}. Our group has shown that periostin is expressed in the developing cardiac valves. Expression begins around E10.5 in the mouse and HH22 in the chick in the endocardial cushions as the endothelium is transforming into migratory mesenchymal cells. Expression continues throughout valve development showing robust expression in the mature valve fibroblasts. This expression is maintained in the valves throughout the life of the animal. We further determined that extracardiac expression of periostin was evident in fibroblasts present in a variety of connective tissues such as skin, tendon, and bone³⁵. As pertains to this grant, periostin expression is seen in the neonatal cardiac fibroblasts shortly after birth, and subsequently declines to a constant but measurable level that is sustained throughout adult life. Recently, it was reported that periostin is re-expressed during cardiac injury^{31, 38, 39}. However, the mechanism of this regulation and the function of periostin in the injured myocardium has not been described in detail.

How periostin fits into the hierarchy of transcriptional regulation in heart development is unknown. However, we have found that several transcription factors associated with differentiation of cardiac cushion (valvular) mesenchyme have binding sites in the periostin promoter. These include twist, Id2, scleraxis, smads, Msx 1 and Tbx 20. In vitro and in vivo periostin promoter analyses have suggested that critical elements for driving endocardial cushion expression of a lacZ transgene include binding sites for the ubiquitous YY1 transcription factor⁴⁰. However, the expression data reported does not sufficiently recapitulate in situ and immunohistochemical data reported by us, and others; suggesting that other enhancer elements are required for proper recapitulation of periostin expression.

Generation of Periostin Knock Out Mice: To examine the function of periostin in vivo, periostin knockout mice were generated and will be used extensively in this project. The details for creating these mice and their genetics are described in^{31, 35}. These mice display an inhibition in the differentiation of embryonic cushion cells into valve fibroblastic cells^{37, 41}. Additionally, the atrioventricular (AV) and outflow tract (OFT) valves display abnormal differentiation into cardiomyocytes and osteocytes, respectively^{37, 41, 42}. In the adult heart, FACS analysis has demonstrated that 33% of the cells in the heart are unable to be identified³⁸. This population, as described in Section C, may represent a large population of undifferentiated mesenchyme which could not be determined with the same marker antibodies that sufficiently identify 100% of the cells in wild-type hearts. Additionally, we have recently reported that periostin knock-out mice display altered biomechanics and have mechanistically linked this defect to an alteration in collagen fibrillogenesis³⁵. As fibroblasts are historically defined as cells producing type I collagen, we aim to further examine how periostin may affect fibroblast differentiation by affecting collagen expression, organization, and maturation. Together these findings suggest that periostin is needed to promote full and complete differentiation of progenitor cells into fibroblasts and that its absence can result in expression of other mesodermal lineages.

Periostin Expression is Up-regulated in Postnatal Myocardial Remodeling. Remodeling is a normal developmental process which continues during the first couple weeks postnatally in response to hemodynamic changes¹⁻⁴. This active remodeling process can be triggered by biomechanical, electrical or chemical signals. If the modulating signals continue after adaptation to a changing environment, the remodeling process can become detrimental to cardiac function and ultimately lead to irreversible damage and heart failure⁴³⁻⁴⁷. Typically, normal neonatal remodeling is observed as (i) an increase in fibroblast numbers (hyperplasia) and new formation of fibrous ECM components, especially collagen and (ii) hypertophy of existing myocytes and their subsequent exit from the cell cycle. During adult pathological remodeling, as a result of injury, the cardiomyocytes and fibroblasts reactivate this neonatal program. For instance, cardiomyocytes can (i) increase in size (hypertrophy)⁴⁸, (n) undergo apoptosis^{49, 50}, or (iii) revert to a myofibroblastic phenotype⁵¹⁻⁵⁵. A similar neonatal reawakening program is seen for the cardiac fibroblasts, with extensive proliferation and infiltration of the wounded area followed by excessive matrix deposition. It is now well understood that periostin expression is stimulated, in some cases over 60 fold, by cardiac fibroblasts during the injury process⁵⁶⁻⁵⁸. Examining the periostin null mice following injury, three independent groups, including us, reported that scar formation was diminished and over time these mice were more likely to regain normal cardiac function^{31, 38, 39}. Clearly from these pioneering studies, periostin plays a crucial role in promoting myocardial remodeling and is of hallmark importance to collagen fibrillogenesis and scar formation.

B4 Summary

The vertebrate myocardium is composed of cardiac myocytes, non-myocytes and the surrounding ECM. Yet, most studies, while acknowledging the presence of other cell types, have focused on cardiac myocytes as they make up the bulk of the myocardial volume as well as provide the primary mechanical properties (contraction) of the heart. The other cell types, especially cardiac fibroblasts, have received less attention beyond mostly descriptive studies related to their secretion of ECM components. Yet, the number of fibroblasts dramatically change during remodeling (normal and pathological) while the relative number of myocytes remains constant or decreases. When there is a deviation of fibroblast numbers in the heart, a significant physiological response occurs altering the dynamic signaling between the cellular and acellular components of the heart. In this regard, it is significant that we have found, in preliminary studies that periostin, a secreted matricellular protein, is a key candidate regulatory signaling component during these neonatal and pathological remodeling processes. Thus, the proposed hypotheses will test mechanisms by which periostin promotes neonatal and adult pathological myocardial remodeling of the heart.

Nearly one in every five deaths occurs annually due to coronary heart disease with over 1 million new and recurrent coronary attacks per year. Roughly 38% of people who experience a coronary attack in a given year die from it and nearly 16 million victims of angina, heart attacks and other forms of coronary artery disease are still living with this debilitating disease. These statistics demonstrate that coronary heart disease, which causes heart attacks and angina, is the leading cause of death in America⁵⁹. While this is the most prevalent disease there is still very little known regarding treatment. The key players intricate to the progression of this disease are the cardiac myocytes and the cardiac fibroblasts. Most of the work investigating myocardial remodeling post infarct/injury has focused on the role of the myocyte. Due to the adult myocyte being terminally differentiated, its regenerative and/or proliferative capabilities following injury are slight. However, investigators continue to examine possible means for making new myocytes by trying to force cell cylce re-entry. To date, this approach has had limited success. We examine the role of the cardiac fibroblast in myocardial remodeling. Although this cell type has largely been ignored during injury repair, we believe it to be of central importance to adapting the heart to the disease state. Clearly formation of a fibrotic lesion or scar impairs the recovering heart by increasing systolic and diastolic pressures as well as causing arrythimias; all of which contribute to congestive heart failure and eventually death. Thus, if one can modify the type of scar, or therapeutically reduce the scar, then cardiac function should improve. To do this in vivo is obviously a formidable task, however, the absence of periostin affects the formation and/or stability of a scar following cardiac injury. Therefore, as shown in Section C, blocking periostin expression by the cardiac fibroblasts during a heart attack can be a therapeutic intervention. Embodiments of the invention comprise such therapeutics.

### DETAILED DESCRIPTION OF THE INVENTION

C.1 Aim1: Preliminary Data in Support of the Hypothesis that Periostin Promotes Fibrogenesis in the Heart.

Expression analyses: The hypothesis for Aim1 arose from a series of in vivo and in vitro studies on isolated embryonic hearts that began with spatiotemporal microarray analyses that revealed scores of candidates³⁴. One of these, periostin, emerged as a “best” candidate based on its pattern of RNA and protein expression and a search of the literature that revealed it was related to a family of adhesion molecules in insects that were critical for targeting axon-mesodermal cell interactions^{28, 35-37, 60, 61}. We subsequently found that periostin was expressed in cardiac “cushion” mesenchyme (precursors to valve interstitial fibroblasts) and epicardial derived cells (which subsequently differentiate into cardiac fibroblasts). Based on RT-PCR, Western analyses, and immunostaining, we've shown that periostin expression continues at high levels throughout intrauterine life and, important for this proposal, peaks during early neonatal life (4-7 days) and subsequently declines to a constant but measurable level that is sustained throughout adult life. Within the neonatal and adult mouse heart, periostin is expressed exclusively by fibroblastic cells of the cardiac valves and ventricular myocardium (FIG. C-1). Moreover, following cardiac injury, expression of periostin intensifies in the wounded area being highly expressed in the infarct border zone and in the scar itself (FIG. C-1, C-2). Picrosirius and Masson's trichrome validate overlap of expression of periostin with collagen I deposition in the scar (FIG. C-2). This foundational data lends support to the hypothesis that periostin is of functional significance to both neonatal myocardial remodeling and adult pathological remodeling processes. To begin understanding the mechanism(s) by which periostin elicitis its potential pro-fibrosis affects, gene targeted mice were generated.

Generation of periostin knockout mice: The targeted periostin knockout mouse to be used in this study was made by Dr. Jeff Molkentin (University of Cincinnati) and is described in³¹. Western blotting and immunostaining confirmed that periostin protein was undetectable in knock-out mice, including placental tissue, indicating maternal-to-fetal transfer did not occur. Recent studies have indicated that while some periostin null embryos die embryonically by E10.5, the vast majority of null mice live into adulthood. For the purpose of this proposal, we focus on the periostin null mice that survive into adulthood.

Characterization of the cardiac phenotype of adult periostin null mice. Almost 100% of the viable 3 month adult null animals manifested reduced heart size (26%) and abnormally shaped hearts (a rounded apex)³¹. The reason for the reduced heart size and altered shape is unclear. Our tentative suggestion for this morphological defect was that fibroblast numbers, thereby extracellular matrix production, was significantly altered. This theory was based upon a detailed characterization of valve defects present in the null animals that demonstrated periostin was essential for valve fibroblast differentiation³⁷. To test this hypothesis, cell sorting was performed (by our collaborator Troy Baudino, University of South Carolina) on periostin null adult hearts and compared to age matched wild-type mice³⁸. For this, conventional phenotype markers for each cell type found in the wildtype adult heart were used (DDR2-fibroblasts, □-SMA-smooth muscle, □-MHC-myocytes, and CD-31/PECAM− endothelial cells). As shown in FIG. C-3, we were able to identify nearly 100% of the cells in wild-type adult hearts using these markers. However, in periostin null adult hearts, we were unable to identify one-third of the cells with these markers. While the ratio of myocytes to fibroblasts appeared similar between nulls and wild-type hearts, we presume that this large percentage of unrecognized cells is either undifferentiated mesenchyme or their differentiation was altered resulting in cell lineages not normally recognized in adult wild-type heart tissues.

To further determine the potential for periostin to affect fibroblast differentiation, we compared mesenchymal cells isolated from periostin null and wild-type mice, in addition to performing “rescue” experiments. For this study, we isolated atrioventricular mesenchyme from E12.5 periostin null and wild-type mice and placed them in hanging drop cultures for 7 days. In addition, purified protein was added to a subset of the hanging drops to determine the ability to rescue any defects observed in marker expression. After 7 days, the hanging drops were collected, lysed and Western analyses were performed for the fibroblast markers collagen I□I, and I□2. Western analyses demonstrate that, in the case of the periostin null mesenchyme, expression of collagen I is signficantly repressed. Interestingly, this affect is reversible when adding back periostin in the form of purified protein. When assayed, there is a reactivation of collagen isoforms (FIG. C-4). These data demonstrate that presence of periostin is needed for collagen production (a defining marker of fibroblast differentiation and maturation). Although the cell type examined in this assay were atrioventricular mesenchyme, we hypothesize that periostin may be playing a similar role in the cardiac fibroblast differentiation programs and as such, propose to analyze this in Aim 1.

Microarray characterization of changes in ECM gene expression in periostin null mice vs wild-type: The hanging-drop assays indicated that periostin may be important for stimulating more than just collagen I. To initially assess if periostin was affecting the expression of other ECM genes (particularly other collagens), DNA microarray analysis using Affymetrix chips were performed. High quality RNA was collected from ventricular myocardium of embryonic, neonatal and adult wild-type and periostin null animals.

Given that functionally related genes often show coordinately regulated expression⁶², ANOVA was conducted to find genes whose expression pattern closely matched that of periostin in wild-type and null hearts. Forty-nine genes were found whose expression pattern matched that of periostin. Of particular relevance to this proposal, these genes showed a similar, neonatal inductive profile in normal hearts but were diminished or abrogated in periostin-null neonatal hearts. Analysis of these genes and their associated gene ontology revealed that most were fibroblast markers or ECM related genes which were significantly represented (p<0.005). This group included procollagens (I, III V, VI), elastin, lysyl oxidase, Adam26 dysintegrin and a fibronectin type-III domain-containing gene. These findings indicated the importance of periostin in promoting a more global fibroblastic ECM gene expression.

Lack of periostin reduces scar formation after injury. Recent data by us and others have demonstrated that the presence of periostin is essential for scar formation following cardiac injury^{31, 38, 39}. As a corollary, performing either trans-aortic constriction (TAC), ligation of the anterior descending (LAD) coronary, or cryoablation on periostin null mice desmontrated that these animals fail to exhibit significant scar formation 8 weeks post injury (FIG. C-5), thus further substantiating a role for periostin in promoting fibrogenesis.

C.2 Aim2: Preliminary Data in Support of the Hypothesis that Periostin can Influence Ventricular Wall Stiffness

Collagen type I Interactions: The rationale for this hypothesis stemmed from initial work by our group demonstrating that periostin can specifically interact with collagen type I³⁵. Binding of periostin to collagen I was demonstrated by immungold transmission electron microscopy (TEM) on adult heart using specific anti-mouse periostin antibodies and further verified for direct binding to soluble collagen I by immunoprecipitation experiments. To gain a more detailed understanding of how this interactions is mediated, we further propose to identify the specific collagen/periostin binding domain using 55 synthesized peptides and 30 different periostin truncation mutants. All of these reagents have been developed and are currently housed in the applicant's lab. Data generated from these peptides and truncation mutants will provide critical insight into how periostin affects collagen interactions such as cross-linking and maturation and thus, will define novel functional domains within the periostin molecule.

Periostin increases tensile strength of collagen rich tissues. In Aim 2, we propose that periostin is a key matricellular candidate for promoting wall stiffness in adult hearts. To determine the feasibility of testing this hypothesis, we initially measured stress-strain relationships of adult skin specimens which have high expression levels of periostin and collagen. We compared wild-type to null skin samples and found that fibrous tissue from freshly isolated skin of periostin knockout mice exhibited lower tensile strength and modulus of elasticity in contrast to those from the wild-type skin samples³⁵.

Periostin promotes collagen compaction. One of the ways by which periostin can affect tensile strength is by promoting collagen compaction. Of relevance to this proposal, collagen compaction is an essential process that promotes scar formation during pathological remodeling in the adult heart^{5-10, 63-65}. Identifying molecular cues that can stimulate collagen compaction would provide integral data leading to a better understanding of how a scar forms during pathological remodeling. Preliminary data indicate that periostin plays a significant role in promoting collagen compaction. By modulating periostin expression through the use of adenoviruses, compaction of collagen gels by mesenchymal cells and bone marrow stem cells can be modified (FIG. C-6). To determine a possible mechanism, we investigated perisotin signaling through specific integrin receptors expressed by mesenchymal cells (□₁ or □ᵥ/□₃). As detailed in Butcher et al. ⁶⁶we found that periostin induced integrin-mediated collagen compaction through the Rho/PI-3 kinase pathway. In Aim 2, we will seek to determine if periostin can also signal adult ventricular fibroblasts to compact collagen and, if so, what domain of periostin is responsible for the effect, and which integrin is promoting this activity.

C3. AIM3: Preliminary Data in Support of the Hypothesis that Periostin is Expressed by Progenitor (Bone Marrow Derived) Cells and is Necessary for Promoting Fibrogenesis in the Adult Heart.

Bone marrow hematopoietic stem cells (HSCs) give rise to circulating progenitor cells that are recruited into the adult mouse heart and differentiate into new fibroblastic cells. To prove that bone marrow HSCs contribute new fibroblasts to the heart as part of a normal homeostatic process, we employed a clonal HSC transplantation protocol described in five recent publications^{17-19, 67, 68}. When this protocol was combined with the use of clonal bone marrow-derived HSCs from EGFP mice the chimeric mice generated provided a powerful means of evaluating HSC potential. Significant findings from these studies include: evidence that 1) the HSCs give rise to fibroblasts and myofibroblasts in a number of adult tissues including angiotensin-responsive mesangial cells in the kidney, microglial cells in the brain, dermal fibroblasts, and, of major relevance to this proposal, interstitial fibroblasts of the heart^{17, 69-71} (also FIG. C-7), 2) the fibroblastic potential of HSCs is via cell differentiation and not spontaneous fusion of host and donor cells as suggested by some earlier studies^{17, 69-71}, 3) the fibroblastic potential of HSCs is part of normal tissue homeostasis, viz, it is a naturally occurring event (not merely an inflammatory response), 4) the culture system developed for obtaining clonal HSC populations did not alter HSC potential in vivo 5) our transplantation strategy results in long-term, multi-lineage reconstitution of the bone marrow.

Detection of HSC-derived EGFP-positive cells in the adult heart. Our studies using the single cell engraftment assay have revealed an unequivocal contribution of cells of HSC origin to the cardiac interstitial fibroblast population within the ventricular myocardium¹⁸. FIG. C-7 A,B depict EGFP⁺ cells interwoven between cardiac muscle cells in hearts of EGFP⁺ HSC transplanted mice. Given their histological position in the heart and the spindle-shaped morphology of the engrafted EGFP cells, we propose that these cells are fibroblasts or poorly differentiated pre-fibroblasts that are committed to a fibroblast lineage (they also are not recognized by smooth muscle or endothelial markers)¹⁷.

Bone marrow derived cells express type I collagen and the collagen receptor DDR2 in vitro: Bone marrow derived cells in culture express fibronectin, pro-collagen I, vimentin and the discoidin domain receptor 2 (DDR2), a collagen receptor (FIG. C-8), and a marker for cardiac fibroblasts⁷². Because it is known that periostin is strongly up-regulated following ischemic injury, we also sought to utilize our single cell engraftment model to determine which cells actually express periostin. We found that it was the fibroblasts of the post-infarct ventricular scar that were the most intensely positive for periostin. (FIG. C-9). In addition, these cells were defined in the firoblast lineage due to their ability to express type I collagen. This indicates that the fibroblasts of the infarct scar were derived after lethal irradiation from a single HSC and that these cells are the primary source of the elevated expression of periostin seen in this ischemic injury model. Taken together, our studies indicate that HSC-derived cells engraft into the heart and contribute to the cardiac fibroblast population. Based on these findings, the final objective of this application will be to determine whether abrogation of periostin expression will inhibit their differentiation into cardiac fibroblasts

Bone marrow injection of novel periostin shRNA lentiviruses reduces scar formation in vivo. Due to the preliminary data obtained, we decided to test, in vivo, whether abrogation of periostin in bone marrow stem cells followed by cardiac injury would result in a reduced scar and improved cardiac function. For this experiment, we developed and tested a variety of novel shRNA lentiviruses for their effectiveness in in vitro and in vivo assays. From these initial experiments we generated 2 lentiviruses that are capable of completely blocking periostin expression (FIG. D-1). Thus, in conjunction with our collaborator (Dr. Robert Gourdie-MUSC) we performed bilateral injections of one of these periostin blocking shRNA lentiviruses directly into the medullary canal of both femurs in the same mouse. The femur was chosen due to it being the largest bone in the mouse and thus contains the highest density/amount of accessible bone marrow stem cells. Following the injection, the chest cavity was opened and a cryoinjury was given to the mice. Cryoinjuries were chosen as the technique of choice due to their consistent injury size and depth as compared to the LAD and TAC models⁷³. Mice were closed and allowed to recover from surgery. These mice were followed for 6 weeks by echocardiography. As depicted in FIG. C-10, mice that received the periostin blocking lentivirus (experimental animals) showed a significant reduction in scar formation when compared to the controls. Echo data at the one week time-point demonstrated that the degree of the cryoinjury was comparable between the control and experimental animals with a decrease of ˜18% vs. ˜20% in left ventricular ejection fraction respectively after 1 week. However, by the four-week time-point, the experimental animals showed significant increases in overall cardiac function when compared to the controls. When these hearts were sectioned, we found that not only was there a significant reduction in the size of the scar, but there was also indications of myocardial repopulation. As shown in FIG. C-11, myocytes (depicted in red) are found throughout the scar with the highest proportion of these cells near the epicardial border. Since the cryoinjury is obtained by ablating the outer wall of the myocardium, and the demonstration that myocytes are present above the fibrosis zone (blue), we believe these to be newly formed myocytes repopulating the scar formed region. In addition, collagen staining appears significantly more disordered in the periostin “blocked” hearts suggesting that collagen organization is disrupted (data not shown). Whereas much cardiac research is focused on attempting to promote adult myocyte cell cycle re-entry, we chose the novel approach of attempting to modify scar formation in vivo. It is tempting to speculate that, eventhough a scar is formed in both hearts, the one formed in the periostin blocked animals seems to be a “better scar” thereby permitting natural remodeling of this region with new myocytes. These experiments will be expanded (in Aim 3) to utilize the more traditional, established injury models (e.g. TAC) and to further test the efficacy of using these lentiviral transduced bone marrow stem cells as potential therapeutic interventions for scar formation during adult pathological fibrosis.

Section D: Research Design and Methods

D1. AIM1: To determine the mechanism(s) by which periostin promotes fibrogenesis in the heart. By fibrogenesis we mean the formation of fibrous connective tissue as a developmental, reparative or reactive process. Critical for this process to occur is the differentiation of progenitor mesenchymal cells into mature fibroblasts. Admittedly, the fibroblastic phenotype is hard to characterize to everyone's satisfaction. In part, this is because fibroblasts are heterogeneous. For this aim, we will assign a fibroblast phenotype to adult cardiac cells as those that strongly express type I collagen and DDR2 (discoid domain receptor 2) at the mRNA and protein level. The collagen receptor, DDR2, is a particularly reliable marker as it is expressed only by cardiac fibroblasts and never cardiomyocytes. In addition, non-fibroblasts can be identified by their expression of □-MHC (myocytes), □-SMA (vascular smooth muscle cells), or CD31/PECAM (endothelial cells). Based on our preliminary findings that adult periostin null hearts exhibit a significant reduction in cardiac fibroblast numbers and a drastic increase in “unidentifiable” cells, we hypothesize that periostin regulates the differentiation and/or activation of fibroblasts in the mammalian heart. To test this hypothesis, three main experiments are proposed: Experiment 1: To characterize the “unidentifiable” cell population present in the periostin null mice; Experiment 2: As a corollary to experiment 1, we propose to assay the affect of gain and loss of periostin function on adult cardiac fibroblast phenotype; and Experiment 3: To compare proliferation and apoptotic indices between periostin null and wild-type mice in vivo in addition to gain and loss of periostin function in vitro as a possible explanation for reduced fibroblast numbers in the null mice.

Rationale: Numerous studies have documented changes in ECM composition during development and disease⁷⁴⁻⁷⁶. However, it is unknown whether these changes are accompanied by alterations in cell populations. In order for fibrogenesis to occur during cardiac development and disease, differentiation and subsequent proliferation of cardiac fibroblasts as well as their secretion of ECM is of central importance to the remodeling heart. Of clinical relevance, preliminary data presented in Section C demonstrates that following cardiac injury, the periostin null mice have a significant defect in fibrotic remodeling of the injured myocardium resulting in reduced scar formation and enhanced cardiac function. Therefore, understanding mechanisms underlying how periostin mediates cardiac fibroblast activation/differentiation and proliferation should provide significant insight into adult pathological myocardial remodeling processes.

D1.1 Experiment 1: To characterize the “unidentifiable” cell population present in the periostin null mice. Preliminary studies have demonstrated that there are significant differences in the number of fibroblasts in the periostin null animals compared to wild-type. In addition, these studies have identified a large population of cells that are not recognized by conventional markers that are sufficient for recognizing nearly 100% of all cells type in wild-type animals. For this experiment, two main approaches will be undertaken to further examine the identity of these cells: (I) a more comprehensive FACS analysis, in collaboration with Dr. Troy Baudino (University of South Carolina) using other relevant cell markers and (II) to determine the migration and invasion capabilities of these “unidentified” cells as a readout of cell behavior and function.

For the first approach it will be important in this study to determine if this “default population” displays stem cell/macrophage markers and/or retains characteristics of fibroblasts but has simply lost DDR2 expression. To determine if stem cell markers and/or macrophage markers are present in the unlabelled population of cells, we will use the established markers c-Kit, Sca-1, and CD45 for stem cells and F4/80 for macrophages in our FACS analyses. To determine if these cells are still fibroblasts that have lost their DDR2 expression, FACS analyses will be performed using vimentin and fibroblast specific antigen (FSA) (gift from Magdi Yacoub). From our preliminary studies we estimate that a minimum of 10 adult animals, 3-4 months of age for each genotype (periostin null and wild-type) will be used for statistical analyses. Detailed methods for these studies are described below.

For the second approach we will focus on obtaining viable default cells of which we will be able to culture and examine specific cellular behaviors such as migration and invasion. For this, we will cross mice expressing □-MHC-EGFP mice (obtained from Professor Loren Field, IUPUI) onto the periostin null background. FACS sorting will be performed as described below without permeabilization of cells. Thus we will be able to sufficiently sort out DDR2+ fibroblasts, □-MHC-EGFP myocytes, CD31 endothelial cells leaving a highly enriched population of viable “unidentified” cells. Cells will be maintained and expanded in culture for up to 4 passages. To facilitate the phenotypic identification of these cells, migration and invasion assays using standard 3-D collagen gel culture systems will be utilized as described in the protocol below. The degree of cell invasion and migration will be compared to those values obtained for adult wild-type fibroblasts. In addition, we hypothesize that these cells may exhibit more mesenchymal characteristics similar to that of neonatal fibroblasts. Thus, we will compare the degree of migration and invasion of the “unidentified” cells with that obtained for wild-type neonatal cardiac fibroblasts.

D1.2 Experiment 2: To assay the affect of gain and loss of periostin function on adult cardiac fibroblast phenotype. Preliminary data presented in Section C and further examined in Experiment 1 suggested that periostin plays an important role in promoting differentiation of progenitor cells into cardiac fibroblasts. As a corollary to this first experiment, we propose to determine if: (i) blocking periostin can reverse the process of cardiac fibroblast differentiation, and if (ii) over-expression of periostin promotes excessive fibrogenesis and collagen deposition. These approaches will further examine the mechanism(s) by which periostin can promote fibrogenesis. For both approaches, we will utilize periostin expressing adenoviruses (previously verified and published by us) to assay gain of function, and novel periostin shRNA lentiviruses to assay loss of function. To block periostin expression we have developed five novel lentiviral shRNA expressing constructs. Each of these constructs were tested for their efficacy of blocking endogenous periostin in culture. As depicted in FIG. D-1, two of the five lentiviruses (#65, #66) completely abrogated endogenous periostin expression in adult cardiac fibroblasts. These two lentiviruses are directed against different parts of the periostin mRNA (5′ CDS and 3′UTR) and will both be used in all assays described and compared against each other to verify no significant off-target affects. Three main assays to determine gain and loss of periostin function will be performed: (i) FACS analyses, (ii) Western and real-time RT-PCR analyses, and (iii) migration and invasion assays.

FACS analyses will be performed as described below with slight modifications. First, adult wild-type cardiac fibroblasts will be isolated and maintained in culture on 60 mm dishes. After the second passage, the cells will be split onto 150 mM dishes and grouped into three categories: periostin block (using shRNA lentiviruses), over-expression (using periostin expressing adenovirus), and control (using GFP adenovirus). For each of these conditions, viruses (each at a multiplicity of infection of 50—which has been previously determined as optimal for infecting 80-90% of all cells) will be added 24 hours after the split and allowed to infect for 4 days. These time frames have previously been determined as optimal for maximal viral production and knock-down of endogenous periostin protein (FIG. D-1 and). After 4 days, a portion of these cells will be examined for specific cell markers (same markers as described above in Experiment 1) by FACS analyses. Remaining cells that are not used for FACS analyses will be examined by Western blotting and real-time RT-PCR (per standard protocols) for additional markers of fibroblast differentiation including: periostin, collagen I, III, V, VI, vimentin, and DDR2). For the third assay, cardiac fibroblasts will be isolated and placed in hanging drop culture (40,000 cells/20 □l drop). After 16 hours (the minimal time required for this number of cells to form an aggregate) each aggregate will be infected with either the shRNA periostin lentivirus, the periostin expressing adenovirus, or a control GFP adenovirus. 24 hours after infection, cells will be placed onto collagen hydrogels and migration and invasion assays will be performed and measured as detailed below.

D1.3 Experiment 3: To compare proliferation and apoptotic indices between periostin null and wild-type mice in vivo in addition to gain and loss of periostin function in vitro as a possible mechanism for reduced fibroblast numbers in the null mice. Periostin null mice exhibited a significant reduction in cardiac fibroblast number. In addition to affecting differentiation of progenitor cardiac fibroblasts (Experiments 1 and 2), we will assay whether the decreased number of fibroblasts is partly due to a reduction in proliferation and/or an increase in apoptosis. For these studies we will perform in vivo labeling of proliferating cells via intraperitoneal injections in wild-type and periostin null mice in combination with dual FACS analysis as described below. We will further examine the affect of gain and loss of periostin on cardiac fibroblasts proliferation and apoptosis in vitro using the viruses and culture conditions as described in Experiment 2. Infected cells will be also assessed for proliferative and apoptotic indices via FACS analysis as described below.

D1.4 Materials and Methods

Isolation of Cardiac Cells

Adult and 2 week-old neonatal animals will be sacrificed via cervical dislocation and whole hearts will be isolated at indicated time points. Hearts will be flushed with ice-cold Moscona's solution, immersed in Krebs-Ringer Buffer I (KRB) (136 mM NaCl, 28.6 mM KCl, 1.9 mM NaHCO₃, 0.08 mM NaHPO₄, pH 7.4, +BSA 1 mg/ml), homogenized and canulated using a 19-gauge needle. The homogenate will be incubated at 37° C. with shaking at 100 rpm for 5 min. The supernatant will be collected and remaining tissue immersed in 6 ml KRB II (KRB+BSA 2 g/ml+collagenase 140 U/ml), canulated and incubated at 37° C. while shaking for 10 min. This process will be repeated seven times, with the supernatant being collected and pooled with that of prior steps. The pooled supernatant will be centrifuged at 1,000×g for 5 min at 4° C. Cells will be resuspended in 1 ml FACS staining buffer (PBS w/out Mg or Ca²⁺, 1%, FBS, pH 7.4) and counted using a hemocytometer. Cells will be resuspended at 1×10⁶ cells/50 □l for FACS analyses.

Antibodies and FACS Analyses

Antibodies to DDR2 (Santa Cruz, sc-7555), □-myosin heavy chain (□-MHC) (Abcam, ab15), □-smooth muscle actin (□-SMA) (R&D Systems, MAB 1420) and CD31 (Zymed, 37-0700) will be conjugated to Q-dots using Q-dot conjugation kits (Invitrogen) and then used in FACS analyses. Cells will be fixed and stained using the BD Cytofix/cytoperm kit (BD Bioscience Pharmingen, San Diego, Calif.) as described by the manufacturer. Briefly, cells will be harvested as described above and stained for DDR2, CD31, □-MHC, □-SMA or for all four antibodies together. For internal markers, cells will be permeabilized using BD-cytofix/cytoperm solution for 20 min at 4° C. and then incubated with antibodies. Cells will be resuspended in FACS staining buffer and analyzed using the Epics XL FACS (Beckman Coulter, Miami Fla.). Stem cells and possible progenitor cells will be stained with c-Kit, Sca-1, CD45, vimentin, and FSA as described above.

3-D Collagen Gel Migration and Invasion Assays

These assays are standard in the applicants lab and have been published^{33, 66}. Briefly, cardiac fibroblasts are isolated and dispersed as described above and divided into three aliquots of DMEM supplemented with 20% FBS and pen/strep. Cells are then aggregated by hanging drop (40,000 cells each) for 16 hours. The aggregates are infected with the three viruses described above and left in hanging drop cultures for an additional 24 hours. Drops are then placed on top of type I collagen gels (1.5 mg/ml) and allowed to adhere for 4 h before 400 μl of additional culture medium was added (DMEM, pen/strep). Fibroblast migration and invasion proceedes for 72 h, after which the cells are fixed with 100% methanol overnight. Gels are then rinsed in 90%, 80%, 70%, and 50% methanol in PBS for 30 min each under gentle rocking, followed by 100% PBS overnight. Cell invasion is quantified manually at 80 μm depth into the gel and at 20 μm intervals deeper until no cells are observed. A minimum of 10 migration and invasion assays will be performed per viral treatment to generate statistically significant values

Proliferation and Apoptosis Assays

Proliferation rates will be determined using a BrdU Flow Cytometry Kit (BD Bioscience Pharmingen) as described by the manufacturer. Briefly, in vivo labeling will be preformed via intraperitoneal injection of 100 □l of 10 mg/ml BrdU. Animals will be sacrificed 16 hrs post injection and hearts will be extracted and cells will be isolated as previously described. Cells will be resuspended, incubated with antibody against DDR2 (fibroblasts), washed and collected by centrifugation. Cells will be permeabilized and stained with antibodies against □-MHC (cardiomyocytes) along with FITC anti-BrdU antibody as described by manufacturer. Washed cells will then be resuspended in 7-amino-actinomyosin D (7-AAD) and analyzed by dual parameter FACS. For in vitro studies, cells will be labeled with 10 □M BrdU for 60 min, harvested and then analyzed as above.

Apoptotic rates will be measured by staining using Ethidium-monoazide (EMA) along with the BD Cytofix/Cytoperm kit. Briefly, EMA diffuses into cells which are undergoing apoptosis and intercalates into DNA. Exposure to light covalently binds EMA to DNA and prevents it's leakage out of the cells. Cells that are not undergoing apoptosis or have compromised membranes (those undergoing necrosis) do not take up EMA. Cells will be stained with EMA under foil for 10 min on ice, labeled with antibody for DDR2, washed, permeabilized and stained with antibody against □-MHC. Samples will be analyzed by FACS as described.

For proliferation and apoptosis statistics, a minimum of 10 mice of each genotype (wild-type and null) will be analyzed for in vivo experiments. For in vitro studies, a minimum of 10 separate, independent isolations and viral treatment regimens will be analyzed to generate statistically significant values as determined by student's t-test and ANOVA analysis.

D.1.5 Anticipated Outcomes, Potential Problems, and Alternative Strategies.

Anticipated Outcomes: Outcomes from Aim1 are anticipated to shed significant light on the mechanisms driving cardiac fibroblast differentiation in the heart. Significant preliminary findings from the periostin null mice have strongly suggested that this ECM protein is important for promoting fibroblast differentiation. Additional previous work from our lab has demonstrated that periostin plays a key role in fibroblast differentiation and maturation of the cardiac valves. In fact, the null mice exhibit a significant reduction in collagen I synthesis that is able to be rescued with exogenous addition of purified periostin protein. In addition, not only do these mice have defects in collagen production and organization, but in the absence of periostin, ectopic cardiomyocytes appear in the forming atrioventricular valve leaflets further suggesting that this gene plays a hierarchical role in directing differentiation of progenitor cells into a fibroblast lineage in lieu of alternative mesodermal cell types (e.g. myocytes). Although we realize that it is a bit naïve to assume periostin is a “do-all” protein, we believe that the microenvironment that periostin helps establish in the extracellular milieu is of hallmark importance for regulating cellular differentiation. Since fibroblast differentiation and the matrix that is produced are of central importance, the data obtained from this aim is expected to shed significant light on normal myocardial remodeling (as occurs during neonatal development) and pathological fibrosis in the adult. Preliminary data (Section C) demonstrated that periostin is expressed intensely in the cardiac fibroblasts during neonatal timepoints and may be functioning as a structural and signaling molecule to help adapt the neonatal heart to the significant increase in hemodynamic pressure during this transition from fetal to adult life. Of clinical significance, periostin expression is “reawakened” during adult cardiac pathologies and may be serving a similar function during adult pathological myocardial remodeling as it did during neonatal development. The outcomes from this initial proposed experiments aim to provide a mechanism for how periostin can promote fibrogenesis in the normal heart which may be applied to models of adult injury as discussed in Aims 2 and 3.

Potential Problems and Alternative Approaches: The main issue that may arise is the inability to sufficiently identify the phenotype and/or molecular make-up of the “unidentifiable” cells in the periostin null mice using the markers as described. As an alternative, more intensive approach to more thoroughly characterize the phenotype of these cells, we have begun to perform microarray analyses of these cells. These data, when compared to normal cardiac fibroblasts will provide a gene profile or composite of these cells. In addition, preliminary data suggests that a neonatal program may be re-activated, thus we are also in the process of performing additional microarray experiments comparing these unidentified cells with cells derived from neonatal hearts to determine if these cells have maintained their progenitor cell status. Pilot studies using FACS analyses on wild-type neonatal hearts have defined a 10-15% population of cells that cannot be identified as myocytes, smooth muscle, fibroblast, nor endothelial cells. We interpret these cells as evidence for an undifferentiated or poorly differentiated population of progenitor cells. Microarray analyses comparing the genetic make-up of these neonatal cells with the unidentified population of cells in the adult periostin null hearts will provide further information as to their phenotype.

Additional approaches currently being pursued by the applicant involves alternatives to those proposed in Experiment 2 which involves gain and loss of function analyses. As experiment 2 is primarily in vitro, we are currently in the process of generating transgenic collagen I□I promoter mice driving expression of periostin specifically in fibroblasts. The collagen I□I promoter mouse has been well documented as specifically driving in the fibroblasts and as such would be an extremely useful tool to examine the potential of periostin to promote excessive fibrosis/fibrogenesis when over-expressed in vivo⁷⁷. Ideally, the periostin promoter would be the best tool to generate over-expressing mice. However, the promoter, as it has been described does not sufficiently recapitulate periostin expression in vivo⁴⁰, and when transfected in primary cardiac fibroblasts in vitro is not active. Of note, we have recently identified a periostin enhancer that appears to contain the necessary elements to drive cardiac fibroblast expression in vitro. We are in the process of generating periostin enhancer-EGFP mice to assess the ability of these sequences to drive expression in cardiac fibroblasts. If these transgenic mice recapitulate endogenous periostin expression then these enhancer elements will be used for periostin over expression studies in vivo.

A final additional approach to be considered for this aim involves alternative migration assays. Scratch assays are a standard two-dimensional in vitro “wounding” assay whereby a small scratch is introduced into a confluent monolayer of cells and the time the cells take to invade and fill in the empty area provides a measure of migration⁷⁸. Thus, these assays will be employed to assess the 2-dimensional migratory capabilities of the “unidentified cells”. The time it takes for “unidentified” cells to fill the gap will be compared to that of wild-type cardiac fibroblasts. Preliminary data on fibroblasts has been previously performed and serves as a benchmark for comparison (FIG. D-2). This 2-D culture assay will serve as a nice complement to the 3-D migration and invasion assay proposed in Experiment 2.

As all techniques are standard in the applicant's lab, no technical issues are expected to arise. If unforeseen technical issues were to arise, other departmental faculty whom are experts in cellular, molecular, and developmental biology will be consulted.

D2. AIM 2. To determine the mechanism(s) by which periostin can influence ventricular wall stiffness. The overall hypothesis proposed is that periostin can increase ventricular wall stiffness by promoting changes in: (i) fibroblast-ECM attachment (ii) compaction of interstitial collagen fibrils, and (iii) collagen organization. This aim relates to how periostin can affect fibroblast-matrix interactions within the heart and how these interactions equate to tissue biomechanics (i.e. ventricular wall stiffness)

Rationale: Interactions between the cardiac fibroblasts and the ECM are essential in maintaining normal cardiac performance, especially during the first two weeks after birth when the mouse ventricular myocardium undergoes significant increases in wall stiffness. As collagen comprises ˜85% of the ventricular myocardial ECM, factors that regulate collagen deposition and organization in neonatal hearts or during pathological remodeling of injured adult hearts is a major question of intense current interest with enormous clinical relevance but few answers. Our preliminary and published data indicate that periostin is a good candidate for examining mechanisms driving collagen mediated ventricular wall stiffness because it has been shown by our group to (i) specifically bind to type I collagen, (ii) promote collagen synthesis/stability and (iii) be involved in promoting collagen cross-linking. For this aim, we hypothesize that periostin can promote, directly or indirectly, ventricular wall stiffness through (i) promoting cardiac fibroblast adhesion, (ii) stimulating collagen compaction, and (iii) promoting collagen organization. Three main experiments are proposed:

D2.1. Experiment 1: To test the hypothesis that cardiac fibroblasts interact with periostin through specific integrin receptors to promote their adhesion. Previous data by our group has demonstrated that mesenchymal cells adhere to periostin substrate via specific integrin receptors⁶⁶. As the majority of this work has been performed on atrioventricular cushion/valve mesenchyme, it will be important to define whether the cardiac fibroblast is capable of these similar interactions. In addition, the data obtained from this experiment will shed significant light on mechanisms underlying cardiac fibroblast interactions with the extracellular milieu and how this may relate to stabilizing the myocardial wall during active remodeling processes. For this assay we will employ three main approaches: (i) determine the ability for periostin to promote fibroblast adhesion and compare this with other known adhesive proteins found in the neonatal and adult heart (collagen I, III, V, VI, fibronectin⁷⁹⁻⁸⁵); (ii) to define the cell surface integrin(s) necessary for periostin mediated adhesion; (iii) to define the specific region within the periostin molecule responsible for this interaction. For each of these three approaches, both neonatal and adult wild-type cardiac fibroblasts will be tested. The reason for incorporating neonatal fibroblasts is due to the proposed theory by others that during adult injury, cardiac fibroblasts may assume a more fetal or neonatal gene program. Thus examining neonatal cardiac fibroblast adhesion characteristics may, in turn, provide important information as to how the adult fibroblast behaves during myocardial injury. The protocol for isolating cardiac fibroblasts is detailed in Section D1.4 and works equally well for isolating either neonatal or adult cardiac fibroblasts.

(i) To test periostin and collagen I, III, V, VI, and fibronectin for their adhesive properties, wells will be coated with titrating amounts of each of these proteins with dilutions of 80, 40, 20, 10, 5, 2.5, 1, 0.5, 0.1 □g/ml. These amounts are empirically chosen based on previously reported data by us and others as being an appropriate range to see a titrating affect in adhesion. Purified periostin will be purchased from RandD sytems (eukaryotically produced full length mouse protein) whereas all other purified proteins will be obtained from Rockland, Inc. After coating the dishes, wells are blocked in 1% BSA for 1 hour and wild-type neonatal or adult cardiac fibroblasts (40,000 cells) are allowed to adhere for 2 hours followed by fixing and staining of cells with 10% ethanol/0.2% crystal violet. Dye is then released from the cells in a 1:1 100% ethanol: phospate buffer, pH4.5 and quantitated using a UV microplate reader (BIO-TEK Instruments) at OD₅₅₀. These experiments will be performed in triplicate and repeated a minimum of 5 times for each titrated amount of purified protein and compared to a 1% BSA negative control to generate statistically significant values. To verify specific interactions additional adhesion assays will be performed as previously described with one slight modification. Prior to plating on each of the matrix coated dishes, the cells will be pre-incubated with each of the matrix molecules. This should function to specifically block binding of the cells to their substrate. These “control” experiments will also be performed in triplicate and repeated a minimum of 5 times.

(ii) To define the cell surface integrin receptors that may be promoting cardiac fibroblast adhesion to periostin, we will initially coat dishes with periostin protein as previously described. Prior to plating the cells, integrin antibodies (□V/□3, □1, □V/□5, □V, □3, and □6 from chemicon) will be incubated with the cell suspension for 30 mintues and cells will be tested for adhesion as previously described. To validate specific interactions with defined integrins, immunoprecipitation (IP) pulldown experiments will be performed as previously described by us³⁵.

(iii) To define the specific region of periostin that is required for cardiac fibroblast adhesion, 54 overlapping peptides corresponding to the full-length periostin protein have been synthesized and are currently being tested. These peptides are represented in FIG. D-3. These peptides (100 □M) will be coated onto 96-well dishes and adhesion assays will be performed as described above. To verify specific interactions with the cardiac fibroblast, candidate adhesive peptides will be incubated with the cells prior to plating on dishes containing full length purified protein. In addition, integrin blocking experiments (as described above) will be performed to identify and further validate specific interactions with the receptors.

D2.2. Experiment 2: To test the hypothesis that periostin mediates/stimulates cardiac fibroblasts compaction of collagen fibrils. Rationale: Fibroblasts in the highly ordered, endomysial collagen network surrounding ventricular myocytes can generate mechanical force by unified compaction^{63, 84, 86, 87}. The compaction of collagen within the heart has been shown to be dependent on PI3 and Rho kinase signaling pathways and is exacerbated by TGF□3 stimulation. Importantly we have obtained preliminary data using chick mesenchyme that link each of these pathways or signaling mechanisms to periostin⁶⁶; thereby further substantiating our hypothesis that periostin can increase wall stiffness by promoting collagen gel compaction. The major goal for this Aim is to determine the mechanism(s) by which periostin can promote ventricular myocardial wall stiffness. Examining if, and how, periostin promotes compaction of collagen into densely organized fibrous bundles will provide us with a mechanistic explanation of how periostin influences myocardial wall stiffness. We will examine the affects of periostin on fibroblast mediated collagen compaction and decipher if this process is occurring through an integrin dependent signaling mechanism. Additionally, to further define the functional domains within the periostin protein, we will test the 54 various periostin peptides, as described in Section D2.1, for their ability to promote fibroblast mediated collagen compaction.

Experimental Approach: Collagen compaction assays will be performed as previously described by us. Briefly, neutralized collagen monomers will be prepared (1.5 mg/ml) will be mixed with 1×10⁵ neonatal or adult cardiac fibroblasts and neutralized with 0.15N NaOH to form solidified fibrous networks by 1 h in culture at 37° C., after which 400 □l of culture medium is added. Collagen gels are released from the walls of the wells after 12 h, and allowed to compact freely for 7 days. In all experiments, collagen gel compaction will be quantified by measuring the ratio of compacted area to the day 0 original area. All compaction experiments are performed in triplicate and repeated a minimum of 3 times.

Is collagen compaction modified in the periostin−/− background? Specifically, we will perform collagen gel compaction assays using wild-type and periostin null cardiac fibroblasts from neonatal and adult hearts which are entombed within collagen gels. After 7 days of incubation, the compaction area is measured and compared to the initial starting area. In addition, these gels will be processed for Western analyses and IHC using antibodies specific for □-SMA, vimentin and the various integrins as described in Section D2.1. □-SMA and vimentin were specifically chosen due to these proteins being important in cytoskeletal arrangement and also give an indication of cell contractility with an increase in expression of these proteins indicating enhanced contractility^{88,89}. In addition to testing wild-type and null cardiac fibroblasts, gain of function experiments will also be performed. For this, titrating amounts of purified periostin or each of the 54 synthesized periostin peptides (10 □g/ml) will be incorporated into the gels prior to polymerization. Both wild-type and periostin null cardiac fibroblasts will be tested with titrating amounts of periostin (or the peptides) to determine if (i) exogenous addition of periostin (or periostin peptides) enhances collagen compaction and (ii) compaction defects determined in the null fibroblasts can be “rescued” with either full length periostin or a peptide porition of the protein.

Does periostin dependent integrin binding stimulate intracellular signaling to promote collagen compaction? Integrin dependency on collagen compaction will be determined by identical experiments in the presence of blocking antibodies to either □ᵥ/□₃ and/or □₁integrins (5 μg/ml) as described above for the periostin null and wild-type neonates and adult fibroblasts. Effects of Rho kinase and PI 3-kinase on collagen compaction will be determined by identical experimentation by the addition of small molecule inhibitors Y-27632 (Calbiochem, 5 μM) or wortmannin (Calbiochem, 1 μM) to the media, respectively. The extent of collagen compaction will be defined as described above and Western analyses of compacted gels will be examined for changes in □-SMA and vimentin expression. This approach has been very successful in defining pathways through which periostin signals in atrioventricular mesenchyme previously published by our group⁶⁶ and will further define the mechanism(s) by which periostin can affect collagen compaction.

D2.3. Experiment 3: To test the hypothesis that periostin is crucial for proper collagen matrix organization and overall tissue properties. Hydroxyproline comparative analyses between age-matched wild-type and periostin null left ventricles indicated no significant difference in collagen content either in the neonatal or adult hearts (data not shown). However, preliminary data presented in Section C and published by others suggested that ventricular wall stiffness was significantly reduced in the null mice. This suggests that periostin does not have a significant affect on regulating collagen I synthesis, but plays more of a crucial role in promoting the organization of a collagenous network to ensure greater tissue stability. Thus, in this experiment, we examine the mechanism(s) by which ventricular wall stiffness may be affected by periostin expression. For this we will undertake three approaches comparing wild-type to periostin null hearts by investigating collagen organization and how this may ultimately affect ventricular wall strength. The first two approaches examining collagen fibril diameter and cross-linking are both good indicators of collagen fibrillogenesis and may provide a mechanistic understanding for how periostin can regulate ventricular wall strength⁹⁰.

(i) Collagen fibril diameter present in the left and right ventricles will be examined and compared between the adult null and wild-type mice by transmission electron microscopy (TEM). Alterations or redistribution of collagen fibrils is used as an indicator of aberrant collagen fibrillogenesis. This will be accomplished as we have previously published. Briefly, left ventricles from wild-type and periostin null mice will be isolated and immersed in 2% glutaraldehyde. Samples are rinsed and post-fixed in 1% osmium tetroxide in 0.1M coadylate buffer, and thereafter dehydrated in graded acetones and embedded in Epon. Ultrathin section are stained with 3% uranyl acetate and 0.2% lead citrate, and examined under a JEOL electron microscope. At least 4 mice of each genotype will be used for measurement and collagen fibril diameter and distribution. Micrographs from non-overlapping regions of the left ventricle are taken from cross-sections. The distribution of collagen fibril diameters are calculated using NIH image. 8 areas in a square are chosen for determination of distribution.

(ii) The degree of collagen fibril cross-linking will be measured by differential scanning calorimetry (DSC). Fibril cross-linking provides a measure of collagen fibril assembly and maturation and will provide insight into how periostin may be affecting ventricular wall strength. These experiments will be performed essentially as we have previously reported³⁵. Briefly, portions/segments (5 to 8 □g) of freshly isolated hearts from a minimum of 5 periostin null and wild-type mice are sealed in aluminum pans and their DSC thermograms are recorded on a Mettler Toledo DSC 822e calorimeter with temperature increments of 5° C./min. For the hearts, a minimum of 5 pieces of the left ventricle, right ventricle and inter ventricular septum will be tested from each heart to determine the potential of chamber specific differences.

In addition to testing the ability for periostin to facilitate collagen cross-linking, we propose to additionally define the collagen interacting domain within the periostin protein. This will be done by utilizing specific “FLAG Tagged” periostin carboxyl truncation mutants. Each of these deletion mutants have been generated and tested for their ability to express the appropriately sized periostin piece in HEK293 cells (FIG. D-4). Immunoprecipitation pull-downs experiments will be performed as we have previously published for the full-length periostin/collagen interaction.

(iii) To investigate the influence of periostin on cardiac tissue properties, biomechanical analyses (i.e. stress-strain curves, ultimate stress, and incremental modulus of elasticity) will be performed and compared between wild-type and periostin null mice. For these, hearts will be excised from wild-type and periostin null (N=6) mice. Tensile tests will be performed by initially cutting transverse myocardial rings of approximately the same size. The specimens are kept in M199 media at room temperature and tested within 1 hour of sacrifice. Before measurements are obtained, myocardial rings are placed between two microscope slides and overall thickness of the specimens is measured with a micrometer device “Ultra Digital Mark IV” (Fowler, Swiss) with accuracy of ±0.001 mm. Specimens are then gripped between specially designed alligator clamps to prevent slippage, and tensile tests are performed using MTS materials testing system (Synergie 100) with a load cell of 50N. Force elongation curves are recorded at a constant elongation rate of 5 mm/min until failure. During experimentations, the heart specimens are kept continuously moist with room temperature M199 media. Stress is calculated from force in Newton (N) divided by the initial cross-sectional area of the specimen. Incremental modulus of elasticity between the levels of stress 0.25/0.3 MPa of samples are calculated also using MTS provided software.

D.2.5 Anticipated Outcomes, Potential Problems, and Alternative Strategies. Recent work by REFS has demonstrated that the ventricular walls of the periostin null mice are significantly weaker than that of the wild-type counterparts. However, the mechanisms by which periostin can affect ventricular wall strength has not be explored. Our recent report has suggested that one of the mechanisms by which periostin could affect the material properties of the ventricular myocardium is through playing an important role in collagen fibrillogenesis. The three experiments proposed in this aim will examine if, and the extent to which, collagen fibrillogenesis is altered in the myocardium of the periostin null mice. All of the techniques proposed in this aim have been tested in other system/tissues by the applicant and, as such, no major technical issues are anticipated to occur. However, it is possible that, unlike other connective tissues, periostin may not have a detectable affect on collagen fibrillogenesis. Based on current literature surrounding periostin, this too would be a surprising, yet significant finding as it would help define interesting differences between how the processes of collagen maturation/fibrillogenesis occurs in the ventricular myocardium versus other connective tissues.

The use of peptides in Experiments 1 and 2 are expected to yield exciting data pertaining to the specific region within the periostin molecule that binds to integrins and stimulates a cellular response. However, it is possible that the native conformation of the periostin protein brings together two or more non-overlapping regions that are necessary for integrin binding. In this case, the peptides will be ineffective in defining crucial interaction domains. To address this potential concern, we have generated 10 additional epitope tagged constructs as described in Section D2.4. Following transfection of HEK293 cells, we have been able to “pull down” the “FLAG” tagged proteins from serum-free media using anti-FLAG agarose beads and purify these constructs to near homogeneity. If necessary, these purified truncation mutants will be utilized in cases where peptides are ineffective in demonstrating and affect.

In many cases, matricellular proteins like periostin exhibit more noticeable affects during active remodeling processes including regeneration or remodeling following injury^{91,92}. Thus, as an alternative to the experiments proposed, we will also perform an analysis of collagen fibrillogenesis following myocardial injury. For this, we will utilize two main approaches of injury of the periostin null and wild-type ventricular myocardium: trans-aortic constriction and cryoablation. Both of these injury models have distinct benefits and when tested in parallel can provide a wealth of information. Whereas the TAC model will generate general left ventricular fibrosis, the cryoinjury can produce a scar of defined area and depth. We will analyze the degree of scar formation in both the wild-type and periostin null animals. In fact, preliminary data in Section C indicate that 8 weeks following cryoablation the size of the scar in the periostin null mice is significantly reduced indicating that collagen deposition, organization, and/or maturation of fibrils has been altered. To get an idea of how the collagen is organized in these scars three main approaches as described above will be performed: (i) transmission electron miscroscopy to get a measure of fibril diameter, (ii) differential scanning calorimetry to examine collagen cross-linking, and (iii) MTS using strips of fibrotic myocardial tissue to determine the tensile strength of the actual scar. As an additional alternative experiment to examine the affect on collagen fibrillogenesis, it would be interesting to perform cell free turbidity assays to determine if periostin, in the absence of other matrix components, can promote collagen polymerization. Although the turbidity assays will not definitively provide specific information as to how periostin may be affecting ventricular wall stiffness, the assay may provide additional information about periostin regulation of collagen fibrillogenesis.

D3 Aim 3: To Test, In Vivo, the Hypotheses that Bone Marrow Derived Stem Cells Contribute to the Cardiac Fibroblast Population and Expression of Periostin by These Cells Promotes Fibrogenesis During Normal (Neonatal) and Pathological (Adult) Myocardial Remodeling.

Rationale: The overall goal of this aim is to determine why fibrosis (but not myocardial survival or renewal) is enhanced in chronic models of pathological myocardial remodeling. In this context, we propose to test the hypothesis that periostin signaling is essential for promoting the differentiation of cells that are added to the postnatal heart into a cardiac fibroblast lineage. We also seek to determine if loss of periostin signaling leads to alteration of cardiovascular histology, biomechanics and function in both normal and pathological states. Significant to this hypothesis is our preliminary data that indicate that hematopoetic stem cells (HSCs) engraft into the myocardium of recipient mice where they exhibit a fibroblastic phenotype (see Section C). Further, we have shown that hematopoietic stem cells (HSCs) give rise to at least four populations of cells that exhibit fibroblastic properties. In light of our collective findings, we hypothesize that HSC-derived progenitors may be a novel and renewable source of cells that contribute to the cardiac fibroblast population during normal neonatal and adult tissue homeostasis. Experimentation outlined in this Aim is designed to characterize the effects of periostin on differentiation of circulating HSC-derived progenitors to the fibroblast lineage and the effects of abrogation of periostin expression on the differentiation of bone marrow HSC-derived progenitor cells and their response to injury. For this aim, four main experiments are proposed.

D3.1. Experiment 1: Evaluate Whether Ventricular Fibroblasts are Derived Postnatally and Throughout Adult Life for Normal Tissue Homeostasis Through the Recruitment of Circulating Progenitor Cells of Bone Marrow Origin (i.e. from Hematopoietic Stem Cells).

Rationale: The current understanding of the origins of cardiac fibroblasts is that they are derived from embryonic tissues including the endocardium and epicardium (as discussed in Secion B and reviewed in⁸). In light of our preliminary studies, which detect HSC-derived cells with fibroblastic morphology in the ventricles of adult recipient mice, experiments proposed in Experiment 1 of Aim 3 are designed to assess bone marrow HSC-derived cell contribution to the cardiac fibroblast population during neonatal and adult tissue homeostasis.

To determine the contribution of cells of bone marrow HSC origin to the cardiac fibroblast population (which undergoes expansion during the first three weeks of neonatal life) we have developed a perinatal donor HSC transplantation strategy. The importance of this approach is that it will permit us to quantify absolute numbers of HSC-derived fibroblasts that engraft into the cardiac tissues during neonatal remodeling of the myocardium as well as selected points during adult tissues homeostasis. To accomplish this, we combine perinatal busulfan conditioning with IP injection of lineage marked (EGFP⁺) donor bone marrow cells on day 1 of neonatal life^{17, 18, 93, 94}. Using this strategy, we have demonstrated contribution of EGFP bone marrow cells to the cardiac valve leaflets¹⁷

Determining the bone marrow-derived: endogenous fibroblast ratio: Mice exhibiting a high level of EGFP cell engraftment will be sacrificed at 1, 3, 6 and 12 months post-transplantation and the number of bone marrow derived fibroblasts (EGFP⁺/DDR2⁺ cells) will be compared to endogenous fibroblasts (EGFP⁻/DDR2⁺ cells) by FACS analysis. As discussed previously, DDR2, discoidin domain receptor 2, is a collagen receptor that we have found to be expressed specifically by fibroblasts. Ratios of bone marrow-derived to endogenous fibroblasts will be determined by flow analysis of both populations to detect EGFP⁺/DDR2⁺ and EGFP⁻/DDR2⁺ cells as previously described in Section D1.4.

ECM biosynthesis: After the quantitative contribution of bone marrow cells to the cardiac fibroblast population is established, we will confirm these results in situ by detecting bone marrow derived (EGFP⁺) fibroblasts using antibodies to DDR2. We will further confirm the fibroblastic nature of EGFP⁺/DDR2⁺ cells by using in situ hybridization to detect collagen I, III, V, VI, and periostin mRNA in conjunction with IHC for DDR2 (on the same section). This is a technically feasible approach and dual immunolabeling/ISH has been previously published by our group.

Rates of proliferation and apoptosis: Cell proliferation and death will be evaluated in situ in paraffin sectioned tissues using routine methods described previously (Section D1.4). EGFP expression will allow us to distinguish bone marrow derived fibroblasts from other fibroblasts and provides a means of comparing cell proliferation and death rates between bone marrow-derived and endogenous populations. Briefly BrdU (5-Bromo-2′deoxyuridine, 200 mg/kg) will be IP injected into EGFP BMC transplanted mice daily for three days to label dividing cells. Hearts from these mice will be harvested after five days (at the time points listed above), fixed, and immunolabeled using rat anti-BrdU antibodies (Accurate Chemical and Scientific, Westbury, N.Y.) to detect proliferating cells. We will confirm BrdU labeling studies by additionally utilizing phosphorylated histone H3 (H3P) antibody that labels condensed DNA (M phase of mitosis). Two apoptosis detection methods will be used: the Apoptag® kit (Serologicals, Norcross, Ga.) to detect fragmented DNA and antibodies that recognize cleaved caspase-3 (detect cells prior to death).

D3.2 Experiment 2: Determine the in vivo effects of inhibiting periostin expression on the contribution of bone marrow progenitor cell contribution to the cardiac fibroblast population. We hypothesize, based on our preliminary findings, that loss of periostin signaling will result in decreased differentiation of circulating bone marrow-derived progenitors to the cardiac fibroblast population in vivo. If true, additional assays to further assess whether these changes were sufficient to alter the biomechanical properties of the myocardial wall will be undertaken (as described in Aim 2).

To investigate the effects of loss of periostin expression on the contribution of bone marrow-derived progenitors to the cardiac fibroblast population of recipient mice, we will perform neonatal transplantation (described in D.3.1) of EGFP periostin^{-/-} bone marrow cells into both wild type (periostin^{+/+} EGFP⁻; cohort1) and periostin^{-/-} EGFP⁻ (cohort 2) recipient mice, and periostin^{+/+} (wt) EGFP bone marrow cells into periostin^{+/+} EGFP⁻ recipients (cohort 3). The three experimental cohorts will permit us to examine the question of ‘seed versus soil’ in our investigation of the role of periostin in differentiation of bone marrow HSC-derived cells to the cardiac fibroblast lineage. To accomplish the transplantation step of Experiment 2, we will bring both recipient and donor mice onto the same background. Cardiac tissues will be collected at the same time points as in D.3.1 (1, 3, 6 and 12 months). Differences in contribution of periostin^{-/-} and periostin^{+/+} cells to the cardiac fibroblast population of recipient mice by FACS analysis will be quantified and the fibroblastic nature of EGFP⁺/DDR2⁺ cells will be confirmed using in situ hybridization to detect collagen I and periostin mRNA (described in D.3.1). We will also evaluate the effects of loss of periostin expression in cells of bone marrow origin that engraft into cardiac tissues on biomechanical wall strength. This will be accomplished using unfixed, freshly isolated hearts subjected to the biomechanical tests described for Aim 2 and previously published by us. Additional hearts will be analyzed by cross sectional analysis for differences in wall thickness, collagen cross-linking and altered ECM deposition, as described in Aim 2. Six (6) experimental mice and corresponding control mice (N) will be collected and analyzed for each timepoint. Each experiment will be repeated three times.

D.3.3 Experiment 3: Determine whether fibrosis and pathological remodeling are affects of periostin mediated progenitor cell recruitment and differentiation following cardiac injury. Our preliminary studies demonstrate that HSC-derived cells are responsive to myocardial injury (FIG. C-9). Thus, we reason that the signals elicited by pathological remodeling of the injured myocardium increase the recruitment of circulating HSC-derived cells and that these cells will participate in the deposition of fibrous ECM in the injured myocardium. Therefore, the specific question in Experiment 3 is whether loss of periostin expression by cells of bone marrow origin that migrate to the injured myocardium will result in decreased fibrosis. The overall hypothesis for this experiment, based on preliminary data, is that loss of periostin expression will result in a reduction in fibrogenesis by: (i) reducing total numbers of HSC-derived fibroblasts in the injured myocardium and/or (ii) suppress differentiation of HSC-derived progenitors into cardiac fibroblasts.

Wild-type recipient mice will be transplanted with HSCs from EGFP⁺:PN^{+/+} mice (to permit distinction of bone marrow-derived fibroblasts from endogenous fibroblasts). Highly engrafted recipient mice will be identified as previously described. These mice will be subjected to TAC (trans-aortic constriction) induced cardiac hypertrophy, and analyzed at 3, 7, 15, and 30 days post-injury. Cardiac tissues from TAC and sham-operated control mice will be harvested and processed for FACS quantification of global ratios of bone marrow-derived to endogenous fibroblasts. This will be accomplished by flow analysis of both cell populations (EGFP⁺/DDR2⁺ and EGFP⁻/DDR2⁺) as described above in D.3.1. Fibroblast numbers from injured hearts will be statistically analyzed against control (sham-operated animals) within the cohort. Numbers of EFGP+ fibroblasts within the injured myocardium will be quantified as follows: every 10^{th} 3 mm section thru the injured region of the ventricular myocardium will be immunolabeled with anti-DDR2 and anti-GFP. Total numbers of DDR2⁺/EGFP⁺ and DDR2⁺/EGFP⁻ cells will be counted (Photoshop). Total numbers of HSC-derived (EGFP⁺) fibroblasts will be expressed as the percentage of total fibroblasts in the injured region of the myocardium. We will confirm the fibroblastic nature of EGFP⁺/DDR2⁺ cells as described in D.3.1). To test the corollary hypothesis that loss of periostin signaling will modulate fibroblastic differentiation of recruited HSC progenitor cells and result in reduced fibrosis, both wild-type (cohort 1) and periostin−/− (cohort 2) recipient mice will be transplanted with donor HSCs from EGFP⁺:periostin−/− mice. Highly engrafted mice from both cohorts (plus a control cohort of wild-type mice transplanted with wild-type bone marrow cells) will be subjected to TAC. We will harvest the cardiac tissue at the same timepoints post-injury (day 3, 7, 15, 30) and process as described above. Functional analyses, followed by morphometric and histological analyses will be performed similarly to those described in D3.1. We will also evaluate the effects of loss of periostin expression in cells of bone marrow origin that engraft into cardiac tissues on ventricular wall stiffness. This will be accomplished using unfixed, freshly isolated hearts subjected to biomechanical assays described in Aim 2. Additional hearts will be subjected to morphometric and histological analyses for differences in collagen deposition and regional differences in wall thickness as described in Aim 2. To accomplish this, the left ventricle will be dissected free of the rest of the heart and the area of injury will be dissected from the remaining LV under aseptic conditions and processed separately for assessment of collagen content. Collagen isotype-specific changes will be assayed by qRT-PCR. In addition to functional assessment and morphometric/histological analyses of the injured zone(s), we will determine whether periostin−/− HSC-derived cells decrease fibroblast differentiation using marker antibodies and procedures described in Aim 1 combined with laser scanning confocal analysis to assay colocalization of EGFP fluorescence.

D3.4 Experiment 4: To determine whether specifically blocking periostin expression in bone marrow stem cells abrogates pathological fibrosis following myocardial injury. Preliminary data and experiments laid out in this Aim and the previous aims have led us to performing this in vivo approach for abrogating periostin expression in bone marrow stem cells. For this highly experimental approach, we perform bilateral bone marrow injections of shRNA periostin blocking lentiviral constructs directly into the medullary canal of both femurs of wild-type mice. The femurs are chosen since they contain the highest proportion of stem cells and are the most accessible for in vivo viral delivery. The femur injections are immediately followed by the generation of a myocardial injury using either cryoablation or TAC models. The overall goal of this experiment is to test whether genetically modifying bone marrow stem cells (as occurs with the lentiviral constructs) to inhibit periostin production in vivo will abrogate fibrosis following injury and directly or indirectly enhance cardiac function. Preliminary data presented in Section C strongly suggests that these in vivo viral injections block a fully organized scar from forming and stabilizing, return cardiac function to baseline, and may promote new myocardial growth. This extremely novel, radical approach to modifying stem cell behavior in vivo presents the first known potential treatment for myocardial infarctions through reducing fibrogenesis (scar size) and enhancing cardiac function. By using the stem cell as a delivery vehicle, we hypothesize that these cells infiltrate the wounded myocardium but are unable to activate a fully functional fibrogenic process due to the inhibitory periostin lentiviruses and, as such, results in a reduction in fibrosis, disorganized collagen, and myocardial growth.

It should be noted that this is a highly experimental approach, and to verify to completeness the efficacy of this therapy would certainly encompass many more years of testing and support beyond the timeframe of this proposal. However, as a prelude to those studies, we simply aim to determine: (i) the number of stem cells that were infected in the femurs that actually make it to the injury area, and (ii) their ability, once at the injury site, to repress periostin. For both of these pilot experiments, new lentiviral constructs will be generated. Constructs will be similar to the shRNA lentiviruses currently utilized in these studies with one major difference. These lentiviruses will be engineered to also include an IRES-RFP element so that the infection and transduction of viruses can be followed. After further validation of these constructs in vitro (as previously detailed in Secion C), bone marrow injection/myocardial injury experiments will be performed in the context of irradiated mice that were engrafted with EGFP marked stem cells as described above. After 1,4, 6, and 8 weeks hearts will be excised, fixed, sectioned, and examined by direct fluorescence for presence and number of RFP/EGFP positive stem cells. IHC for periostin on these sections will be performed and compared with control injected bone marrow. In addition, sections will be stained for MF20 and connexin-43, to immunohistochemically myocytes and Masson's trichrome to histochemically define collagen and cardiac muscle. Quantitative confocal microscopy techniques and 3-D reconstructions of immunohistological sections will be performed to validate degree and extent of the stem cell mediated periostin repression as well as to measure potential myocyte re-population.

D3.5 Anticipated Outcomes, Potential Problems, and Alternative Strategies. Anticipated outcomes of Experiment 1 involves the establishment and the extent of bone marrow stem cell progenitor contribution to the cardiac fibroblast population over time. Techniques described are routinely used in our laboratory and the laboratory of our collaborator, Dr. Richard Visconti. We predict no difficulty accomplishing this aim. Based on our preliminary findings, we expect that bone marrow-derived fibroblasts will represent a distinct population whose numbers within the normal myocardium will remain constant, perhaps owning to a cycle of cell death and replacement by newly engrafted cells. For Experiment 2: If our hypothesis is correct that abrogation of periostin expression will result in decreased differentiation of circulating bone marrow-derived progenitors to the cardiac fibroblast population, we expect to see a reduction of HSC-derived (EGFP⁺/DDR2⁺) fibroblasts in recipient hearts. Furthermore, loss of profibrotic cells in the myocardium should also lead to less organized extracellular milieu (i.e. less fibrosis) where these cells engraft, giving a less organized appearance to the ECM and thereby reduced biomechanical function. We predict no difficulties with engraftment of periostin^{-/-} bone marrow cells since periostin^{-/-} mice exhibit no known hematological disorders. For Experiment 3: The proposed studies have a number of possible outcomes. These include: 1) periostin−/− HSC-derived cell engraftment into wild-type, injured hearts results in an amelioration of post-injury pathological changes in myocardial function and histology. In this scenario, lack of periostin synthesis and secretion by HSC-derived cells leads to decreased fibroblast differentiation by recruited HSCs. This, in turn, ameliorates scar formation and perhaps myocyte apoptosis or induces myocytic differentiation of HSC progenitors that ultimately leads to improved cardiac function; 2) Periostin−/− HSC-derived cells engraft into periostin-null hearts but do not ameliorate the post-injury pathological changes. In this scenario, PN−/− cells engraft into the injured region and respond to periostin independent external cues, adopt a fibroblastic phenotype and contribute to post-injury scar formation; 3) Periostin−/− HSC-derived cell engraftment into the periostin-null leads to enhanced pathogenesis. This could occur if loss of periostin expression in the ventricle that is populated by periostin−/− HSC-derived cells leads to a weakening of the injured myocardium; and (4) Periostin−/− HSC-derived cells do not engraft into the injured wild-type myocardium. For scenarios in which we have either improvement or enhanced pathogenesis we will be able to provide mechanistic information for the basis of these outcomes. For Experiment 4: the preliminary findings are quite exciting and the approach is novel for blocking stem cell mediated fibrosis during adult pathological remodeling. As described above, this approach is highly experimental and requires significant additional studies to validate efficacy of this potential therapeutic treatment. Obviously there are many inherent problems that could arise in this experiment and we thoroughly expect there to be mouse to mouse variation in myocardial remodeling due to (i) complications in delivering the lentiviruses to the medullary canal, (ii) infection rate variability, (iii) induction of confounding immune responses following infection, (iv) unforeseen extracardiac side-affects, etc. To circumvent some of these potential issues with invasive bone marrow surgery, we will additionally attempt tail-vein injections of the lentiviruses. This delivery mechanism has the probability of diluting the viruses, but the benefits for the surgery include the ability to inject higher doses over many time periods as well as the procedure being less painful to the mouse. The potential benefits of this technology are greatly significant with this therapy showing promise in promoting “scar less” myocardial regeneration. Key pilot experiments for this promising experiment are described in Section D3.4.

Alternative strategies that could provide important information towards the understanding of how stem cells respond to periostin expression would involve performing in vitro experiments on EGFP+ stem cells. First, using lentiviral constructs already in hand, EGFP+ stem cells are isolated and maintained in culture with the addition of puromyocin. Each lentiviral construct has a puromyocin resistance gene which facilitates the generation of stable cell lines and/or clones. After 2 rounds/passages of puromyocin treatement, highly enriched stably infected stem cells should be obtained. These genetically modified cells are then engrafted back into irradiated mice as previously described above. In a subset of these animals, we will assay for any defects of fibrogenesis (i.e. fibroblast differentiation, biomechanical wall strength, etc.) as described above in D3.2. In addition, a subset of these animals will be subjected to myocardial injury (as described in D3.3) to determine if this genetic manipulation of stem cells is sufficient to generate a phenotypic affect on myocardial remodeling as previously seen for the direct bone marrow injections.

D.4. Time-frame for completion of all projects. All three aims are significantly inter-related. However, the success of each aim does not depend on each other. Therefore, it is conceivable that all three aims could be worked on simultaneously. Based on the number of proposed individuals performing the experiments, we present a realistic time-frame for completion of each experiment and the project as a whole (FIG. D-7).

Delivery Modalities.

Periostin inhibitory reagents may be delivered subcutaneously, transdermally, intravenously, by implantation, by inhalation, by electrical, chemical or physical transduction into tissue, organs and cells and/or bone marrow injection. Delivery of the periostin may include the following delivery methods:
Lenti-Virus
Retroviruses
Adenoviruses (AV)
Adeno-associated viruses (AAV)
Envelope protein pseudotyping of viral vectors (e.g. could use mAb to specifically target the cell)
Non-viral methods
Naked DNA (e.g. transfection via, or)
Oligonucleotides
Lipoplexes and polyplexes (e.g. micelle, liposome, polyplex, etc)
Hybrid methods (e.g. mix of 2 things, such as virosome=liposome and inactivated HIV)
Dendrimers

Embodiments of the invention apply to the novel idea of blocking periostin as a mode of a) promoting tissue regeneration, b) promoting healing, c) curing disease d) inhibiting metastasis, e) blocking scarring, and f) promoting normalized and/or improved physiological state and function. Alternative inhibitory reagents based on the original periostin inhibitory sequence may include:
1. polyethylenimine (PEI)-complexed shRNA;
2. polyethylenimine (PEI)-complexed siRNA;
3. polyethylenimine (PEI)-complexed miRNA;
4. modified RNA sequence: 5′-A.
cₘcₘgggcuaagucuuugcacaguaaacucgaguuuacugugcaaagacuuagcuuuₘuₘuₘgₘ-Chol-3′ B.
cₘcₘggccacaugguuaauaagagaaucucgagauucucuuauuaaccaugugguuuₘuₘuₘgₘ-Chol-3′
   Lower case letters represent 2′-O-Methyl-modified oligonucleotides, subscript ‘m’ represents a phosphorothioate linkage, and ‘Chol’ represents linked cholesterol;
   5. Fusion or combination with membrane permeable or cell permeant chemicals and/or sequences (e.g., polypeptides, lipids, esters) to inhibitor RNA sequences;
   6. Fusion or combination with membrane permeable or cell permeant chemicals and/or sequences (e.g., polypeptides, lipids, esters) to dominant negative periostin polypeptides; and
   7. Targeting by genetic, chemical and other means of upstream modulators of periostin expression or function that would result in a loss of function or expression of periostin in a tissue.

MOA (Mechanism of Action): By blocking periostin, cellular and/or biologic function is affected:
1. within the bone marrow canal;
2. in transit to the injury site;
3. at the site of injury;
4. during transitions between the above loci (e.g., endothelial transmigration); and
5. at alternative sites within the mammal.

### REFERENCES CITED

1. J. P. Guignard, Hypertension in the neonate. Clinical & Experimental Hypertension—Part A, Theory & Practice 8, 723 (1986).
2. J. E. Jones, P. A. Jose, Neonatal blood pressure regulation. Seminars in Perinatology 28, 141 (2004).
3. A. L. Kent, Z. Kecskes, B. Shadbolt, M. C. Falk, Normative blood pressure data in the early neonatal period. Pediatric Nephrology 22, 1335 (2007).
4. A. L. Kent, Z. Kecskes, B. Shadbolt, M. C. Falk, Blood pressure in the first year of life in healthy infants born at term. Pediatric Nephrology 22, 1743 (2007).
5. I. Banerjee, K. Yekkala, T. K. Borg, T. A. Baudino, Dynamic interactions between myocytes, fibroblasts, and extracellular matrix. Ann N Y Acad Sci 1080, 76 (October, 2006).
6. T. A. Baudino, W. Carver, W. Giles, T. K. Borg, Cardiac fibroblasts: friend or foe? Am J Physiol Heart Circ Physiol 291, H1015 (September, 2006).
7. T. K. Borg, J. B. Caulfield, Collagen in the heart. Tex Rep Biol Med 39, 321 (1979).
8. P. Camelliti, T. K. Borg, P. Kohl, Structural and functional characterisation of cardiac fibroblasts. Cardiovasc Res 65, 40 (Jan. 1, 2005).
9. W. Carver, M. L. Nagpal, M. Nachtigal, T. K. Borg, L. Terracio, Collagen expression in mechanically stimulated cardiac fibroblasts. Circ Res 69, 116 (July, 1991).
10. E. C. Goldsmith et al., Organization of fibroblasts in the heart. Dev Dyn 230, 787 (August, 2004).
11. J. M. Perez-Pomares, D. Macias, L. Garcia-Garrido, R. Munoz-Chapuli, The origin of the subepicardial mesenchyme in the avian embryo: an immunohistochemical and quail-chick chimera study. Dev Biol 200, 57 (Aug. 1, 1998).
12. J. M. Perez-Pomares, A. Phelps, M. Sedmerova, A. Wessels, Epicardial-like cells on the distal arterial end of the cardiac outflow tract do not derive from the proepicardium but are derivatives of the cephalic pericardium. Dev Dyn 227, 56 (May, 2003).
13. B. P. Kruithof et al., BMP and FGF regulate the differentiation of multipotential pericardial mesoderm into the myocardial or epicardial lineage. Dev Biol 295, 507 (Jul. 15, 2006).
14. J. M. Perez-Pomares et al., Origin of coronary endothelial cells from epicardial mesothelium in avian embryos. Int J Dev Biol 46, 1005 (December, 2002).
15. J. M. Perez-Pomares, D. Macias, L. Garcia-Garrido, R. Munoz-Chapuli, Contribution of the primitive epicardium to the subepicardial mesenchyme in hamster and chick embryos. Dev Dyn 210, 96 (October, 1997).
16. A. Wessels, J. M. Perez-Pomares, The epicardium and epicardially derived cells (EPDCs) as cardiac stem cells. Anat Rec A Discov Mol Cell Evol Biol 276, 43 (January, 2004).
17. R. P. Visconti et al., An in vivo analysis of hematopoietic stem cell potential: hematopoietic origin of cardiac valve interstitial cells. Circ Res 98, 690 (Mar. 17, 2006).
18. R. P. Visconti, R. R. Markwald, Recruitment of new cells into the postnatal heart: potential modification of phenotype by periostin. Ann N Y Acad Sci 1080, 19 (October, 2006).
19. Y. Ebihara et al., Hematopoietic origins of fibroblasts: II. In vitro studies of fibroblasts, CFU-F, and fibrocytes. Exp Hematol 34, 219 (February, 2006).
20. M. A. Goodell, K. Brose, G. Paradis, A. S. Conner, R. C. Mulligan, Isolation and functional properties of murine hematopoietic stem cells that are replicating in vivo. J Exp Med 183, 1797 (Apr. 1, 1996).
21. M. A. Goodell et al., Dye efflux studies suggest that hematopoietic stem cells expressing low or undetectable levels of CD34 antigen exist in multiple species. Nat Med 3, 1337 (December, 1997).
22. D. Orlic et al., Bone marrow cells regenerate infarcted myocardium. Nature 410, 701 (Apr. 5, 2001).
23. R. S. Schwartz, G. D. Curfman, Can the heart repair itself? N Engl J Med 346, 2 (Jan. 3, 2002).
24. M. Okabe, M. Ikawa, K. Kominami, T. Nakanishi, Y. Nishimune, ‘Green mice’ as a source of ubiquitous green cells. FEBS Lett 407, 313 (May 5, 1997).
25. S. Takeshita, R. Kikuno, K. Tezuka, E. Amann, Osteoblast-specific factor 2: cloning of a putative bone adhesion protein with homology with the insect protein fasciclin I. Biochem J 294 (Pt 1), 271 (Aug. 15, 1993).
26. M. Hortsch, C. S. Goodman, Drosophila fasciclin I, a neural cell adhesion molecule, has a phosphatidylinositol lipid membrane anchor that is developmentally regulated. Journal of Biological Chemistry 265, 15104 (1990).
27. P. M. Snow, A. J. Bieber, C. S. Goodman, Fasciclin III: a novel homophilic adhesion molecule in Drosophila. Cell 59, 313 (1989).
28. C. B. Kern et al., Immunolocalization of chick periostin protein in the developing heart. Anat Rec A Discov Mol Cell Evol Biol 284, 415 (May, 2005).
29. J. Litvin et al., Periostin and periostin-like factor in the human heart: possible therapeutic targets. Cardiovasc Pathol 15, 24 (January-February, 2006).
30. J. Litvin et al., Expression and function of periostin-isoforms in bone. J Cell Biochem 92, 1044 (Aug. 1, 2004).
31. T. Oka et al., Genetic manipulation of periostin expression reveals a role in cardiac hypertrophy and ventricular remodeling.[see comment]. Circulation Research 101, 313 (2007).
32. H. Kudo, N. Amizuka, K. Araki, K. Inohaya, A. Kudo, Zebrafish periostin is required for the adhesion of muscle fiber bundles to the myoseptum and for the differentiation of muscle fibers. Dev Biol 267, 473 (Mar. 15, 2004).
33. K. Inai, R. A. Norris, S. Hoffman, R. R. Markwald, Y. Sugi, BMP-2 induces cell migration and periostin expression during atrioventricular valvulogenesis. Developmental Biology 315, 383 (2008).
34. A. Kruzynska-Frejtag, M. Machnicki, R. Rogers, R. R. Markwald, S. J. Conway, Periostin (an osteoblast-specific factor) is expressed within the embryonic mouse heart during valve formation. Mech Dev 103, 183 (May, 2001).
35. R. A. Norris et al., Periostin regulates collagen fibrillogenesis and the biomechanical properties of connective tissues. J Cell Biochem, (Jan. 16, 2007).
36. R. A. Norris et al., Identification and detection of the periostin gene in cardiac development. Anat Rec A Discov Mol Cell Evol Biol 281, 1227 (December, 2004).
37. R. A. Norris et al., Periostin regulates atrioventricular valve maturation. Developmental Biology 316, 200 (2008).
38. R. A. Norris et al., Neonatal and Adult Cardiovascular Pathophysiological Remodeling and Repair: Developmental Role of Periostin. Ann N Y Acad Sci In Press, (2007).
39. M. Shimazaki et al., Periostin is essential for cardiac healing after acute myocardial infarction. Journal of Experimental Medicine 205, 295 (2008).
40. A. Lindsley et al., Identification and characterization of a novel Schwann and outflow tract endocardial cushion lineage-restricted periostin enhancer. Developmental Biology 307, 340 (2007).
41. S. Snider et al., Periostin is required for maturation and ECM stabilization of the cardiac skeleton. Circulation Research (Submitted), (2007).
42. A. V. Tkatchenko et al., Lack of periostin leads to suppression of Notchl signaling and calcific aortic valve disease. Nature (Submitted), (2007).
43. J. N. Cohn, Critical review of heart failure: the role of left ventricular remodeling in the therapeutic response. Clin Cardiol 18, IV4 (September, 1995).
44. A. M. Deschamps, F. G. Spinale, Disruptions and detours in the myocardial matrix highway and heart failure. Curr Heart Fail Rep 2, 10 (March, 2005).
45. P. W. Fedak, S. Verma, R. D. Weisel, R. K. Li, Cardiac remodeling and failure From molecules to man (Part II). Cardiovasc Pathol 14, 49 (March-April, 2005).
46. P. W. Fedak, S. Verma, R. D. Weisel, R. K. Li, Cardiac remodeling and failure: from molecules to man (Part I). Cardiovasc Pathol 14, 1 (January-February, 2005).
47. P. W. Fedak, S. Verma, R. D. Weisel, M. Skrtic, R. K. Li, Cardiac remodeling and failure: from molecules to man (Part III). Cardiovasc Pathol 14, 109 (May-June, 2005).
48. J. Heineke, J. D. Molkentin, Regulation of cardiac hypertrophy by intracellular signalling pathways. Nat Rev Mol Cell Biol 7, 589 (August, 2006).
49. R. S. Foo, K. Mani, R. N. Kitsis, Death begets failure in the heart. J Clin Invest 115, 565 (March, 2005).
50. R. N. Kitsis, D. L. Mann, Apoptosis and the heart: a decade of progress. J Mol Cell Cardiol 38, 1 (January, 2005).
51. S. Clement, C. Pellieux, C. Chaponnier, T. Pedrazzini, G. Gabbiani, Angiotensin II stimulates alpha-skeletal actin expression in cadiomyocytes in vitro and in vivo in the absence of hypertension. Differentiation 69, 66 (December, 2001).
52. G. D. Dispersyn, M. Borgers, Apoptosis in the heart: about programmed cell death and survival. News Physiol Sci 16, 41 (February, 2001).
53. G. D. Dispersyn, E. Geuens, L. Ver Donck, F. C. Ramaekers, M. Borgers, Adult rabbit cardiomyocytes undergo hibernation-like dedifferentiation when co-cultured with cardiac fibroblasts. Cardiovasc Res 51, 230 (Aug. 1, 2001).
54. G. D. Dispersyn et al., Dissociation of cardiomyocyte apoptosis and dedifferentiation in infarct border zones. Eur Heart J 23, 849 (June, 2002).
55. W. A. Laframboise et al., Cardiac Fibroblasts Influence Cardiomyocyte Phenotype In Vitro. Am J Physiol Cell Physiol, (Jan. 17, 2007).
56. N. Katsuragi et al., Periostin as a novel factor responsible for ventricular dilation. Circulation 110, 1806 (Sep. 28, 2004).
57. P. Li, S. Oparil, W. Feng, Y. F. Chen, Hypoxia-responsive growth factors upregulate periostin and osteopontin expression via distinct signaling pathways in rat pulmonary arterial smooth muscle cells. J Appl Physiol 97, 1550 (October, 2004).
58. L. W. Stanton et al., Altered patterns of gene expression in response to myocardial infarction. Circ Res 86, 939 (May 12, 2000).
59. D. M. Lloyd-Jones et al., Lifetime risk for developing congestive heart failure: the Framingham Heart Study. Circulation 106, 3068 (Dec. 10, 2002).
60. P. Diamond et al., Fasciclin I and II have distinct roles in the development of grasshopper pioneer neurons. Neuron 11, 409 (September, 1993).
61. M. Hortsch, C. S. Goodman, Drosophila fasciclin I, a neural cell adhesion molecule, has a phosphatidylinositol lipid membrane anchor that is developmentally regulated. J Biol Chem 265, 15104 (Sep. 5, 1990).
62. J. L. DeRisi, V. R. Iyer, P. O. Brown, Exploring the metabolic and genetic control of gene expression on a genomic scale. Science 278, 680 (Oct. 24, 1997).
63. T. K. Borg, W. F. Ranson, F. A. Moslehy, J. B. Caulfield, Structural basis of ventricular stiffness. Lab Invest 44, 49 (January, 1981).
64. T. K. Borg, T. Reaves, Cellular Interaction with the ECM during heart development. R. R. a. K. Rubin, Ed., Connective tissue biology: integration and reductionism (Portland Press, London, 1997).
65. T. K. Borg, K. Rubin, E. Lundgren, K. Borg, B. Obrink, Recognition of extracellular matrix components by neonatal and adult cardiac myocytes. Dev Biol 104, 86 (July, 1984).
66. J. T. Butcher, R. A. Norris, S. Hoffman, C. H. Mjaatvedt, R. R. Markwald, Periostin promotes atrioventricular mesenchyme matrix invasion and remodeling mediated by integrin signaling through Rho/PI 3-kinase. Dev Biol 302, 256 (Feb. 1, 2007).
67. A. C. LaRue et al., Hematopoietic origins of fibroblasts: I. In vivo studies of fibroblasts associated with solid tumors. Exp Hematol 34, 208 (February, 2006).
68. V. Mironov, R. P. Visconti, R. R. Markwald, What is regenerative medicine? Emergence of applied stem cell and developmental biology. Expert Opin Biol Ther 4, 773 (June, 2004).
69. D. C. Hess et al., Hematopoietic origin of microglial and perivascular cells in brain. Exp Neurol 186, 134 (April, 2004).
70. D. Mackay, Continuous variation of agonist affinity constants. Trends Pharmacol Sci 9, 156 (May, 1988).
71. M. Masuya et al., Hematopoietic origin of glomerular mesangial cells. Blood 101, 2215 (Mar. 15, 2003).
72. A. Shrivastava et al., An orphan receptor tyrosine kinase family whose members serve as nonintegrin collagen receptors. Mol Cell 1, 25 (December, 1997).
73. H. Huwer et al., Model of chronic systolic and diastolic dysfunction after cryothermia-induced myocardial necrosis in rats. Comparative Medicine 50, 385 (2000).
74. B. C. Berk, K. Fujiwara, S. Lehoux, ECM remodeling in hypertensive heart disease. Journal of Clinical Investigation 117, 568 (2007).
75. Z. Kassiri, R. Khokha, Myocardial extra-cellular matrix and its regulation by metalloproteinases and their inhibitors.[see comment]. Thrombosis & Haemostasis 93, 212 (2005).
76. M. E. Lukashev, Z. Werb, ECM signalling: orchestrating cell behaviour and misbehaviour. Trends in Cell Biology 8, 437 (1998).
77. S. T. Magness, R. Bataller, L. Yang, D. A. Brenner, A dual reporter gene transgenic mouse demonstrates heterogeneity in hepatic fibrogenic cell populations.[see comment]. Hepatology 40, 1151 (2004).
78. C. C. Liang, A. Y. Park, J. L. Guan, In vitro scratch assay: a convenient and inexpensive method for analysis of cell migration in vitro. Nature Protocols 2, 329 (2007).
79. G. G. Ahumada, J. E. Saffitz, Fibronectin in rat heart: a link between cardiac myocytes and collagen. Journal of Histochemistry & Cytochemistry 32, 383 (1984).
80. C. A. Buck et al., Cell adhesion receptors and early mammalian heart development: an overview. Comptes Rendus de L'Academie des Sciences—Serie Iii, Sciences de la Vie 316, 838 (1993).
81. C. Fayet, M. P. Bendeck, A. I. Gotlieb, Cardiac valve interstitial cells secrete fibronectin and form fibrillar adhesions in response to injury. Cardiovascular Pathology 16, 203 (2007).
82. L. Terracio et al., Expression of collagen binding integrins during cardiac development and hypertrophy. Circulation Research 68, 734 (1991).
83. M. J. Loftis, D. Sexton, W. Carver, Effects of collagen density on cardiac fibroblast behavior and gene expression. Journal of Cellular Physiology 196, 504 (2003).
84. S. Kanekar, T. K. Borg, L. Terracio, W. Carver, Modulation of heart fibroblast migration and collagen gel contraction by IGF-I. Cell Adhes Commun 7, 513 (2000).
85. K. S. Masters, D. N. Shah, G. Walker, L. A. Leinwand, K. S. Anseth, Designing scaffolds for valvular interstitial cells: cell adhesion and function on naturally derived materials. J Biomed Mater Res A 71, 172 (Oct. 1, 2004).
86. D. G. Simpson, M. Majeski, T. K. Borg, L. Terracio, Regulation of cardiac myocyte protein turnover and myofibrillar structure in vitro by specific directions of stretch. Circ Res 85, e59 (Nov. 12, 1999).
87. L. Terracio, A. Tingstrom, W. H. Peters, 3rd, T. K. Borg, A potential role for mechanical stimulation in cardiac development. Ann N Y Acad Sci 588, 48 (1990).
88. B. Eckes et al., Impaired mechanical stability, migration and contractile capacity in vimentin-deficient fibroblasts. Journal of Cell Science 111, 1897 (1998).
89. J. T. Butcher, R. M. Nerem, Porcine aortic valve interstitial cells in three-dimensional culture: comparison of phenotype with aortic smooth muscle cells. Journal of Heart Valve Disease 13, 478 (2004).
90. D. L. Christiansen, E. K. Huang, F. H. Silver, Assembly of type I collagen: fusion of fibril subunits and the influence of fibril diameter on mechanical properties. Matrix Biol 19, 409 (September, 2000).
91. P. Bornstein, Matricellular proteins: an overview. Matrix Biol 19, 555 (December, 2000).
92. Z. Yang, T. R. Kyriakides, P. Bornstein, Matricellular proteins as modulators of cell-matrix interactions: adhesive defect in thrombospondin 2-null fibroblasts is a consequence of increased levels of matrix metalloproteinase-2. Mol Biol Cell 11, 3353 (October, 2000).
93. H. Lang et al., Contribution of bone marrow hematopoietic stem cells to adult mouse inner ear: mesenchymal cells and fibrocytes. J Comp Neurol 496, 187 (May 10, 2006).
94. H. Minamiguchi et al., An assay for human hematopoietic stem cells based on transplantation into nonobese diabetic recombination activating gene-null perforin-null mice. Biol Blood Marrow Transplant 11, 487 (July, 2005).
ⁱ Packer M (1989). “Effect of phosphodiesterase inhibitors on survival of patients with chronic congestive heart failure”. Am. J. Cardiol. 63 (2): 41A-45A.
ⁱⁱ Packer M, Carver J R, Rodeheffer R J, et al (1991). “Effect of oral milrinone on mortality in severe chronic heart failure. The PROMISE Study Research Group”. N. Engl. J. Med. 325 (21): 1468-75.

1. We claim the compositions of matter and methods described herein.
